(19) United States
(12) Patent Application Publication
(10) Pub. No.: US 2005/0058983 A1
(43) Pub. Date: Mar. 17, 2005

(54) Use of transgenic mice for the efficient isolation of novel human monoclonal antibodies with neutralizing activity against primary HIV-1 strains and novel HIV-1 neutralizing antibodies

(76) Inventors: Abraham Pinter, Brooklyn, NY (US); Yuxian He, Forest Hills, NY (US); Jose R. Corvalan, Foster City, CA (US)

Correspondence Address: FISH & NEAVE IP GROUP, ROPES & GRAY LLP, 1251 AVENUE OF THE AMERICAS FL C3, NEW YORK, NY, 10020-1105, US

(21) Appl. No.: 10/628,004

(22) Filed: Jul. 25, 2003

Related U.S. Application Data
(63) Continuation of application No. PCT/US02/02171, filed on Jan. 25, 2002.
(60) Provisional application No. 60/264,398, filed on Jan. 26, 2001.
(60) Provisional application No. 60/266,106, filed on Feb. 2, 2001.
(60) Provisional application No. 60/265,984, filed on Feb. 3, 2001.
(60) Provisional application No. 60/270,466, filed on Feb. 21, 2001.

(51) Int. Cl. C12Q 1/70

(52) U.S. Cl. 435/5; 530/388.3

(57) ABSTRACT

The present invention relates to a novel human antibody, and antigen-binding portion thereof, that specifically binds HIV-1 gp120 protein and that has HIV-1 neutralizing activity. The present invention also relates to a cell line that produces an antibody of this invention. The present invention further relates to a pharmaceutical composition or a kit comprising an antibody or antigen binding portion thereof of this invention. The present invention further relates to a method of using the antibody of this invention to treat a subject with an HIV-1 infection or prevent a subject from getting an HIV-1 infection. The present invention also relates to a novel method of making an antibody of this invention. The method involves using a non-human transgenic animal. The present invention further relates to methods of identifying regions of gp120 for use as HIV-1 vaccine.

This invention was made in part with government support under PHS Grant number AI46283 awarded by the National Institutes of Health. The government may have certain rights in the invention.

### TECHNICAL FIELD OF THE INVENTION

The present invention relates to novel antibodies, and antigen-binding portions thereof, that specifically bind HIV-1 gp120 protein and that have HIV-1 neutralizing activity.

The present invention also relates to a cell line that produces an antibody of this invention. The present invention further relates to a composition or a kit comprising an antibody or antigen binding portion thereof of this invention.

The present invention further relates to a method of using the antibody of this invention.

The present invention also relates to a novel method of making an antibody of this invention. In certain embodiments, the method involves using a non-human transgenic animal.

The present invention further relates to methods of identifying regions of gp120 for use as HIV-1 vaccine.

### BACKGROUND OF THE INVENTION

The human immunodeficiency virus 1 (“HIV-1”) is the causative agent for acquired immunodeficiency syndrome (“AIDS”)—a disease characterized by the destruction of the immune system, particularly of CD4+ T-cells, with attendant susceptibility to opportunistic infections—and its precursor. AIDS-related complex (“ARC”)—a syndrome characterized by symptoms such as persistent generalized lymphadenopathy, fever and weight loss.

Despite considerable interest in developing clinically useful monoclonal antibodies (Mabs) against HIV-1, very few such Mabs have been identified. Human monoclonal antibodies (human Mabs) are preferred over rodent Mabs for clinical applications, but isolation of human Mabs by standard methods of EBV transformation of B cells or phage display is inefficient, so that only a small number of human Mabs with neutralizing activity against primary isolates of HIV-1 have been identified. The nature of the antigens used for immunization and screening and the inability to manipulate immunization regimens have also been limiting.

The development of an effective vaccine against HIV has been hindered in part by limited knowledge of the targets on the HIV envelope proteins, gp120 and gp41, that mediate potent neutralization of primary strains of the virus. See. e.g., Cao et al. (1995) N. Engl. J. Med. 332: 201-208; Kostrikis et al. (1996) J. Virol. 70: 445-458; Moog et al. (1997) J. Virol. 71: 3734-3741 and Prince et al. (1987) J. Inf. Dis. 156: 268. While the sera of some infected people contain antibodies that strongly neutralize primary isolates, existing HIV vaccine candidates have not been able to induce similar activities. See, e.g., Berman et al. (1997) J. Infect. Dis. 176:384-397; Bolognesi al. (1998) Nature 391:638-639; Connor et al. (1998) J. Virology 72: 1552-1576; Graham B S et al. (1998) J. Infect. Dis. 177:310-319; Kahn, J. et al. (1995) J. Infect. Dis. 171:1343-1347; Mascola, J. R. et al. (1996) J. Inf. Dis. 173:340-348 and McElrath, M. et al. (1996) Proc. Natl. Acad. Sci. USA. 93:3972-3977. An important approach to identifying such targets is the isolation of Mabs that can potently neutralize viral infectivity. However, despite considerable effort, relatively few Mabs of this sort have been isolated.

Only a handful of human monoclonal antibodies have been described that possess strong neutralizing activities for clinical isolates (Burton, D. R. et al. (1994) Science 266:1024-1027; Moore, J. et al. (1995) J. Virol. 69:101-109; Trkola, A., et al. (1995) J. Virol. 69:6609-6617 and Trkola, A., M. et al. (1996) J. Virol. 70:1100-1108), and as a rule, even these antibodies preferentially neutralized laboratory-adapted T cell-tropic strains over macrophage-tropic isolates. See Honnen, W. J. et al. (1996) p. 289-297, In E. N. F. Brown and D. Burton and J. Mekalanos (ed.), Vaccines 1996: Molecular Approaches to the Control of Infectious Diseases, Cold Spring Harbor Laboratory Press. Combinations of monoclonal antibodies (“Mabs”) have been demonstrated to neutralize synergistically (Vijh-Warrier (1996) J. Virol. 70: 4466-4473; Li et al. (1998) J. Virol. 72:3235-3240), but these effects are relatively modest. The discrepancy between the broad neutralizing capacity of some human sera and the narrower and less potent activities of characterized Mabs suggests that the repertoire of neutralizing epitopes on the surface of clinically relevant HIV-1 strains has not been fully defined.

Most available human Mabs were derived by EBV-transformation of B cells obtained from HIV-1-infected patients, followed by fusion with human-murine heterohybridoma cells, a relatively inefficient process. The neutralizing targets identified in these studies have been fairly limited, and include epitopes in the V3 loop (Conley, A. J. et al. (1994) Proc. Natl. Acad. Sci. USA. 91:3348-3352; Muster, T. et al. (1993) J. Virol. 67:6642-6647; Tilley, S. A. et al. (1992) AIDS Res. Human Retroviruses. 8 :461-467 and Trkola, A. et al. (1995) J. Virol. 69:6609-6617), the CD4-binding domain (Cordell, J. et al. (1991) Virology 185:72-79; Posner, M. R. et al. (1991) J. Immunol. 146:4325-4332; Potts, B. J. et al. (1993) Virology 197:415-419 and Tilley, S. A. et al. (1991) Res. Virol. 142:247-259), a conformational V2 epitope (Gorny et al. (1994) J. Virol. 68:8312-8320); one epitope in gp41 (2F5) (Conley, A. J. et al. (1994) Proc. Natl. Acad. Sci. USA. 91:3348-3352; Muster, T. et al. (1994) J. Virol. 68:4031-4034 and Trkola, A., et al. (1995) J. Virol. 69:6609-6617) and a poorly defined epitope in gp120 (2G12) (Trkola, A. et al. (1996) J. Virol. 70:1100-1108). In addition, two human Mabs have been described that identify conformational epitopes that are induced upon binding of CD4 to gp120 (Thali et al. (1993) J. Virol. 67: 3978-3988), that also have modest neutralizing activities for some isolates. Phage display of recombinant Fabs derived from bone marrow cells of infected patients has allowed the isolation of Mabs directed mainly against the CD4-binding site (Burton et al. (1991) Proc. Natl. Acad. Sci. USA. 88:10134-10137; Ditzel et al. (1995) J. Immunol. 154:893-906; Roben et al. (1994) J. Virol. 68:4821-4828). The most potent and crossreactive of these has been IgGb12, which is directed against a unique gp120 epitope that overlaps the CD4-bs and the V2 domain (Burton, D. R. et al. (1994) Science 266: 1024-1027 and Gauduin et al. (1997) Nature Medicine 3:1389-1393). However, the technical difficulties of this method have limited its widespread application and utility.

### SUMMARY OF THE INVENTION

This invention solves the above-identified problem by providing in some embodiments antibodies, preferably human antibodies, that specifically bind to HIV-1 gp120 protein and that has HIV-1 neutralizing activity, wherein said antibody recognizes (binds) an epitope on a V1/V2 domain of HIV-1 gp120. In some embodiments, said epitope is dependent on the presence of sequences in the V1 loop. In other embodiments, said-epitope is dependent on the presence of sequences in the V2 domain.

This invention also provides an isolated human monoclonal antibody that specifically binds to an epitope on the V3 region of HIV-1 gp120, wherein said antibody does not specifically bind to a peptide consisting of SEQ ID NO: 9 (V3 amino acids 1-20 of the gp120 of HIV-1 MN strain).

This invention also provides a cell line that produces and nucleic acids encoding an antibody of this invention. This invention also provides a pharmaceutical composition and a kit comprising an antibody of this invention.

This invention further provides a method of using an antibody of this invention to treat a subject with an HIV-1 infection. This invention also provides a method of using an antibody of this invention to prevent a subject from becoming infected with HIV-1. This invention further provides a method of using an antibody of this invention to detect HIV-1 infection in a subject.

This invention also provides a method of making human monoclonal antibodies to HIV-1 using a transgenic non-human mammal. In some embodiments this mammal is a transgenic mouse that makes human antibody.

This invention also provides a method of identifying a region on HIV-1 gp120 for use as an HIV-1 vaccine.

The foregoing and other objects, features and advantages of the present invention, as well as the invention itself, will be more fully understood from the following description of preferred embodiments.

### BRIEF DESCRIPTION OF THE DRAWINGS

FIG. 1 Response of XENOMOUSE® Mice to rgp120

FIG. 1A XENOMOUSE® mice immunized with rgp120 developed high titers of anti-gp120 antibodies after immunizations. Serum titers were determined by standard ELISA, using SF162 rgp120 (rgp120_{SF162}) (50 ng/well) as target antigen. Sera from XENOMOUSE® mice were assayed for reactivity with rgp120_{SF162} by ELISA at a dilution of 1/100. Samples were taken three days following the indicated boost with rgp120_{SF162}.

FIG. 1B The ability of XENOMOUSE® mice sera to neutralize HIV_{SF162} was determined following the third boost with rgp120_{SF162}. Neutralization of NL4-3luc virus pseudotyped with SF162 env was determined in U87-T4-CCR5 cells, using serum dilutions of 1:25.

FIG. 2 Initial Mapping of Epitopes Bound by XENOMOUSE® Mabs (Human Mabs from XENOMOUSE® Animals)

ELISA reactivities of XENOMOUSE® Mabs were determined at 10 μg/ml against rgp120_{SF162} before and after reduction with DTT, and against fusion proteins expressing the V1/V2 region of HIV_{SF162} (U.S. Pat. No. 5,643,756, issued Jul. 1, 1997, U.S. Pat. No. 5,952,474, issued Sep. 14, 1999, Kayman, S. C. et al. (1994) J. Virol. 68:400-410 and Krachmarov et al. (2001) AIDS Research and Human Retroviruses Vol. 17, Number 18: 1737-1748; the disclosures of these four references are incorporated by reference herein) or the V3 region of the closely related HIV_{JR-CSF} (Kayman, S. C. et al. (1994) J. Virol. 68:400-410 and Krachmarov et al. (2001) AIDS Research and Human Retroviruses Vol. 17, Number 18: 1737-1748) XENOMOUSE® Mabs are grouped by epitope class, as determined by additional experiments. 8.27.1 and 8.27.3 are derived from two subclones of the original hybridoma clone.

FIG. 3 Mapping of Epitopes in V1 and V2 Domains

XENOMOUSE® Mabs previously scored reactive with the V1/V2_{SF162} fusion protein (U.S. Pat. No. 5,643,756, issued Jul. 1, 1997, U.S. Pat. No. 5,952,474, issued Sep. 14, 1999, Kayman, S. C. et al. (1994) J. Virol. 68:400-410 and Krachmarov et al. (2001) AIDS Research and Human Retroviruses Vol. 17, Number 18: 1737-1748) were retested against this reactivities are presented in FIG. 3A. In FIG. 3B, sequences of the antigens are shown. The sequence (SEQ ID NO: 1) in the fusion protein (“FP”) corresponds exactly to the SF162 isolate, and includes the stem that connects the V1/V2 domain to the core of gp120. The V1 peptides correspond to the SF162 sequence, except that in peptide 130-1 (P130-1) (SEQ ID NO: 2) there is a Ser in place of the Cys N-terminal to the V1 loop, and peptide 130-2 (SEQ ID NO: 3) lacks an R residue that is present in the SF162 sequence (that missing R is between the D residue at position 11 of P130-2 and the G residue at position 12 of P130-2 (SEQ ID NO: 3)). Peptide 130-2 (P130-2) is SEQ ID NO: 3. The V2 peptide (T15K) (SEQ ID NO: 4) corresponds to the sequence of the Case-A2 isolate; two residues that differ from the SF162 sequence are underlined.

FIG. 4 XENOMOUSE® Mabs Neutralization of HIVSF162

Representative neutralization assays of XENOMOUSE® Mabs (filled symbols) and HuMabPs (human Mabs derived from patients) against NL4-3 luc virus pseudotyped with SF162 env, comparing V1 and V2-specific Mabs (FIG. 4A), CD4bs-specific Mabs (FIG. 4B), and V3-specific Mabs (FIG. 4C) (8E11/A8 is a subclone of 8E11).

FIG. 5 Mapping of V1 and V2 Epitopes by Binding Competition

The ability of competing Mabs to inhibit the binding of biotinlyated reagents to rgp120_{SF162} immobilized on ELISA plates was determined. Greater than 40% inhibition of binding was considered positive competition (values in bold). Negative numbers indicate that the indicated percent increase in signal was obtained. Competing Mabs were used at 100 μg/ml.

The molecules that were biotinylated are: 43A3/E4, 35D10/D2, 697D and sCD4 (the first three are antibodies).

FIG. 6 Mapping of V3 Epitopes

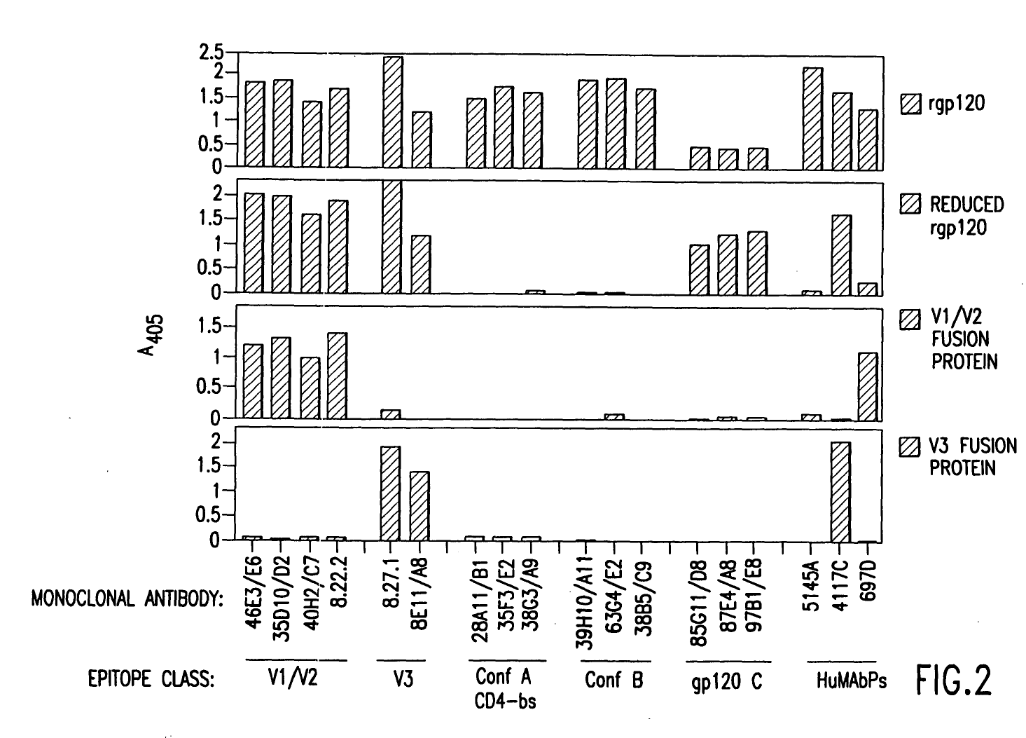

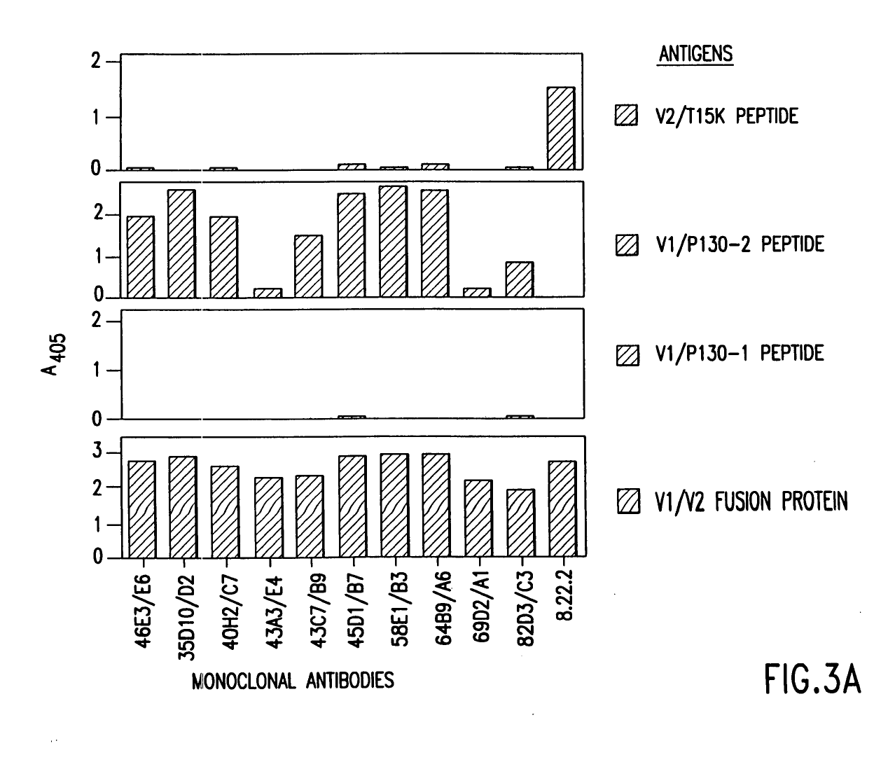

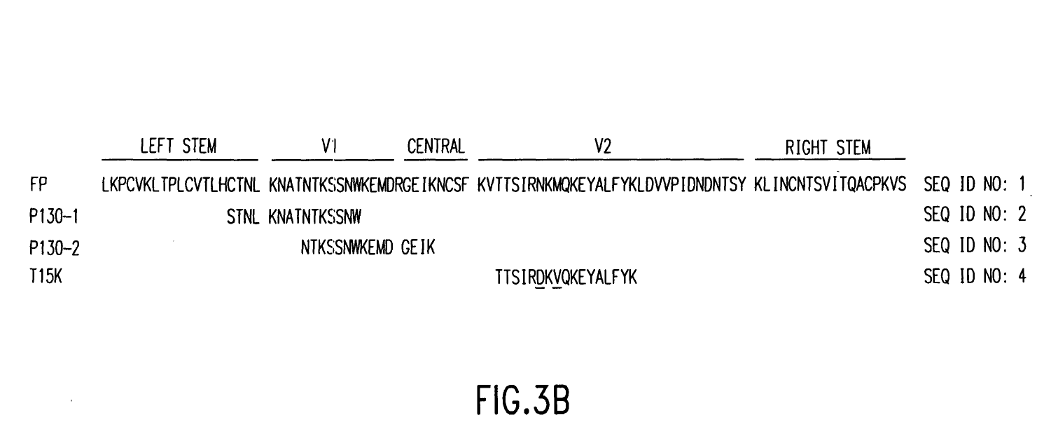

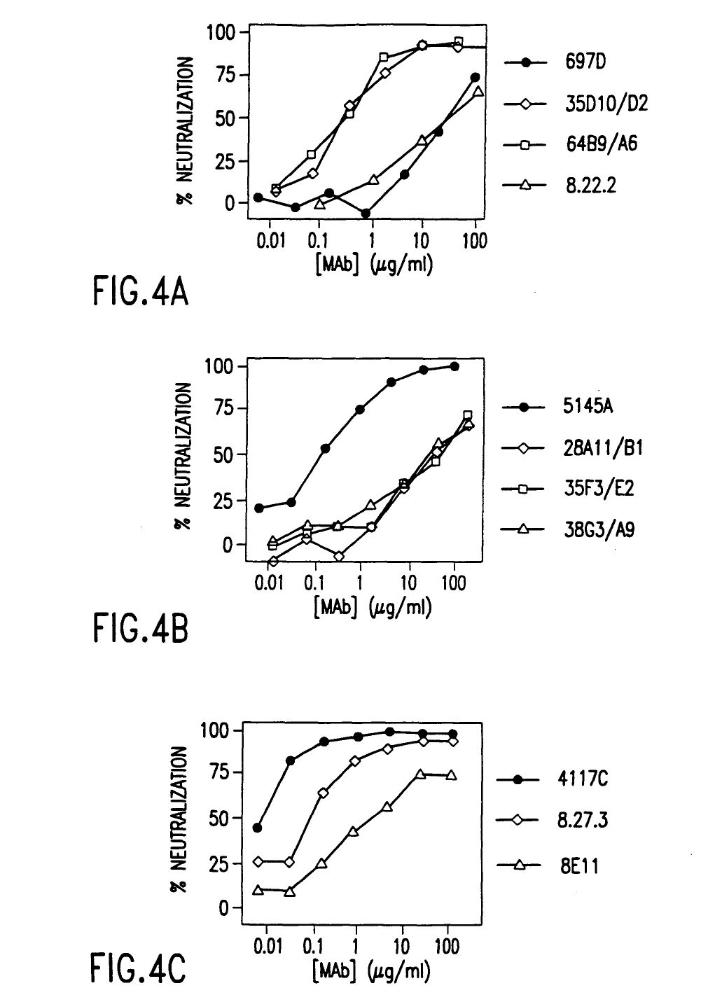

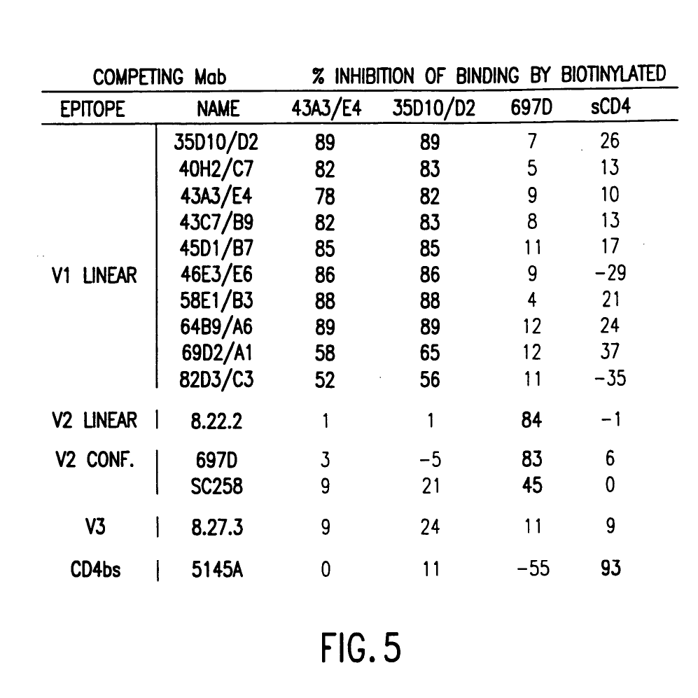

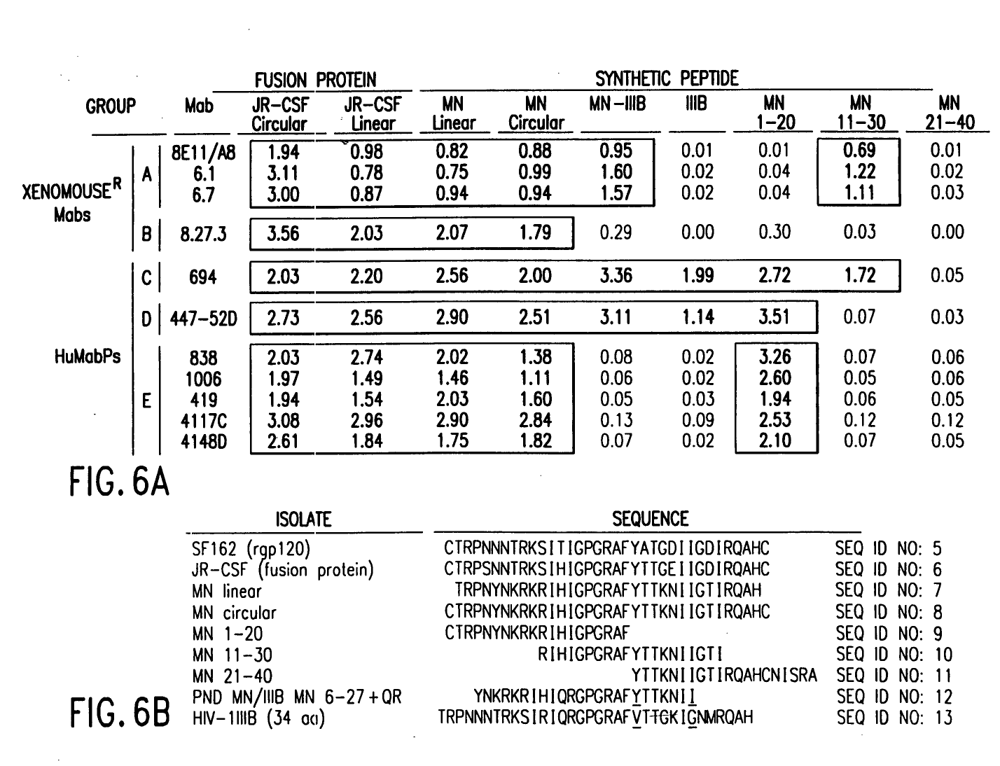

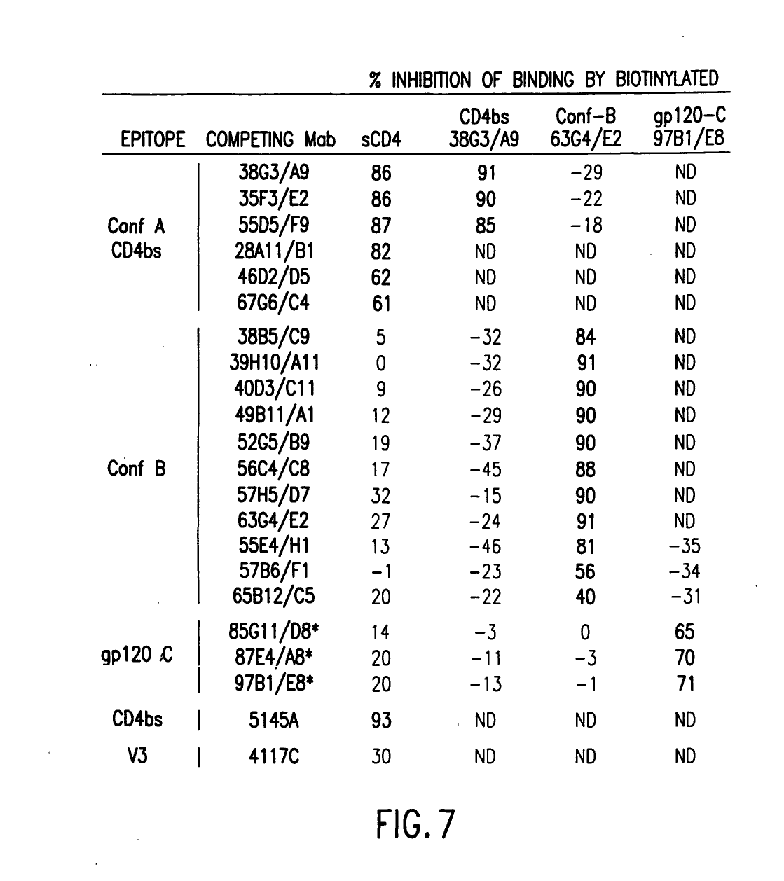

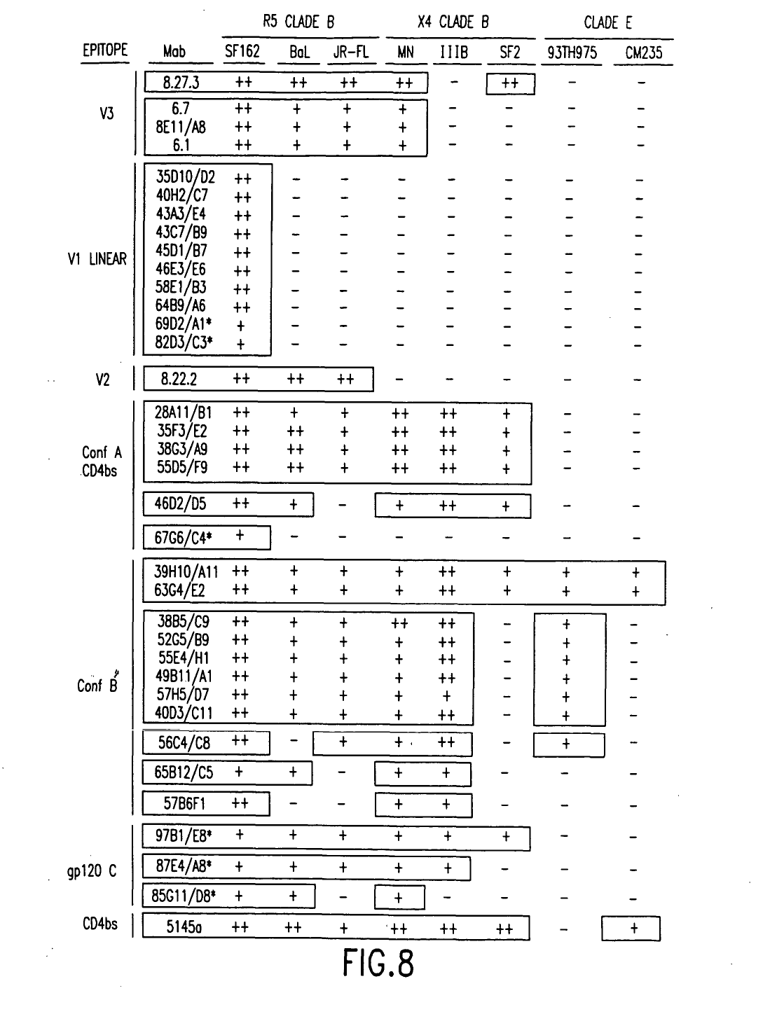

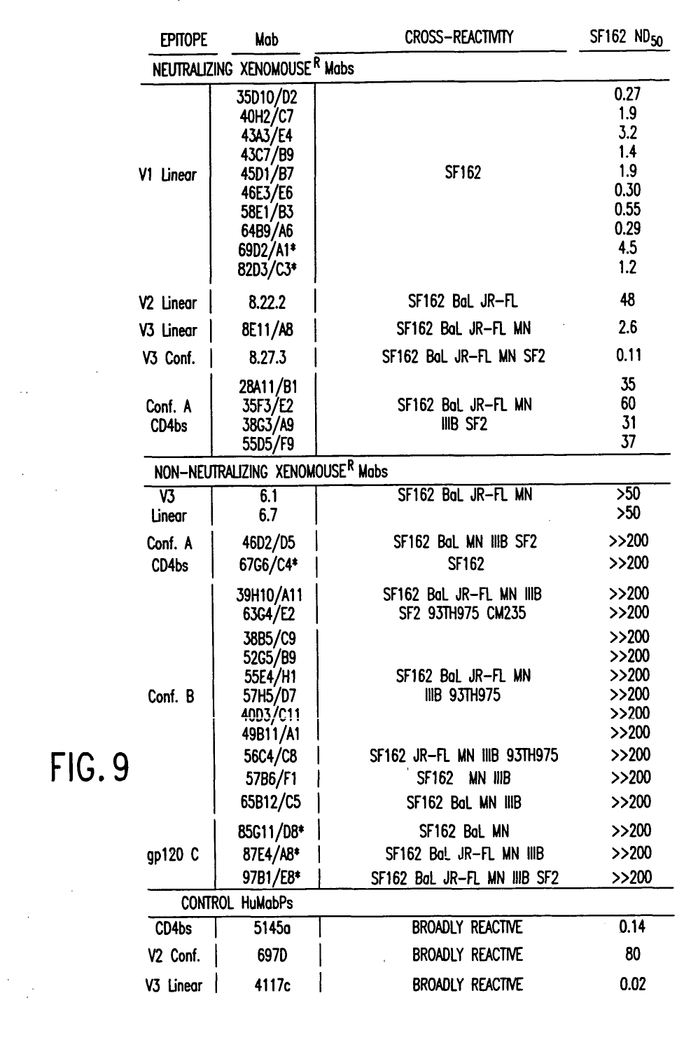

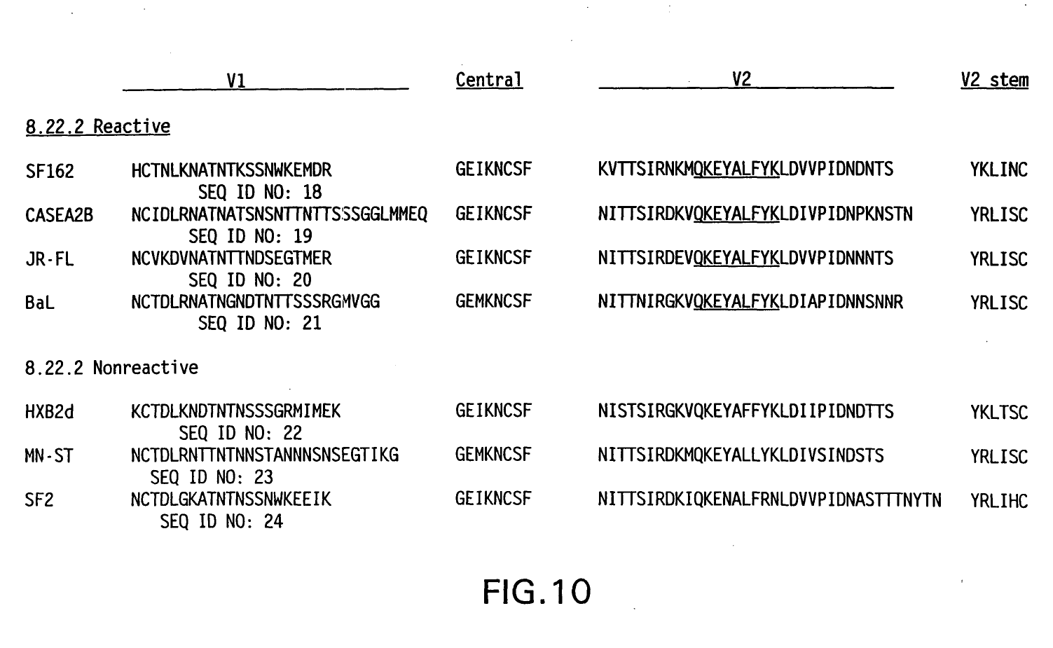

FIG. 6A. The average of duplicate A405 values obtained in the indicated ELISA reaction are presented. Values considered positive are in bold. Fusion proteins at 2 μg/ml and synthetic peptides at 5 μg/ml were used to coat ELISA plates. Mabs were used at 10 μg/ml. Peptide MN-IIIB is PND MN/IIIB MN 6-27+QR (SEQ ID NO: 12) and peptide IIIB is peptide HIV-1IIIB (SEQ ID NO: 13). SEQ ID NO: 5 is the amino acid sequence of the V3 domain vicinity of SF162 (rgp120) and SEQ ID NO: 6 is the amino acid sequence of the V3 domain vicinity of JR-CSF (fusion protein) [JR-CSF (fusion protein) is JR-CSF cirucular and is V3 fusion protein referred to in FIGS. 2-3].

FIG. 6B. Sequences of the V3 loop of HIV_{SF162} and the antigens used in Panel A are aligned. The numbering of HIV_{MN} peptides begins with the N-terminal Cys of the loop. Residues common to Group A-reactive sequences that differ from those of non-reactive HIV_{IIIB} are underlined. The linearized V3_{JR-CSF} fusion protein (JR-CSF linear in FIG. 6) is a mutant V3_{JR-CSF} fusion protein in which the cysteine at the N-terminal base of the V3 loop was mutated to a serine. The V3 domain sequence of JR-CSF linear is STRPSNNTRKSIHIGPGRAFYTTGEIIGDIRQAHC (SEQ ID NO: 27).

FIG. 7 Mapping of Epitopes in Conserved Domains by Binding Competition

The indicated Mabs were tested at 100 μg/ml for the ability to block binding of the indicated biotinylated reagent to rgp120_{SF162} in ELISA. Greater than 40% inhibition of binding was considered positive competition (values in bold). Negative numbers denote that the indicated percent increase in signal was obtained. ND indicates not done. The molecules, that were biotinylated are: sCD4, 38G3/A9, 63G4/E2 and 97B1/E8 (the last three are antibodies).

FIG. 8 Reactivity of XENOMOUSE® Mabs with Diverse rgp120s

The ability of the XENOMOUSE® Mabs and a control HuMabP (5145a) to recognize a series of rgp120s was tested in ELISA. Mabs were used at 10 μg/ml and tested in duplicate. ++ indicates A405s at least tenfold above background, + indicates A405s at least threefold over background (0.24). XENOMOUSE® Mabs isolated following immunization with deglycosylated rgp120_{SF162} are indicated with an *.

57B6F1=57B6/F1. 57B6F1 is another way to write 57B6/F1.

FIG. 9 XENOMOUSE® Mabs Neutralization Activity Against HIV_{SF162}

Neutralization titers against HIV_{SF162} were determined graphically from data such as those in FIG. 4. ND₅₀s are reported in μg/ml; > indicates that 50% neutralization was not reached, and >> indicates that essentially no neutralization was seen, at the indicated highest concentration used. XENOMOUSE® Mabs isolated following immunization with deglycosylated rgp120_{SF162} are indicated with an *.

FIG. 10 shows V2 region sequences of gp120s tested for reactivity with Mab 8.22.2. A sequence present in the region mapped by peptide T15K (SEQ ID NO: 4) that is conserved in the reactive sequences (QKEYALFYK (SEQ ID NO: 26)) is underlined.

(SEQ IN NO: 18)

HCTNLKNATNTKSSNWKEMDRGEIKNCSFKVTTSIRNKMQKEYALFYKLD
VVPIDNDNTSYKLINC.

(SEQ ID NO: 19)

NDIDLRNATNATSNSNTTNTTSSSGGLMMEQGEIKNCSFNITTSIRDKVQ
KEYALFYKLDIVPIDNPKNSTNYRLISC.

(SEQ ID NO: 20)

NCVKDVNATNTTNDSEGTMERGEIKNCSFNITTSIRDEVQKEYALFYKLD
VVPIDNNNTSYRLISC.

(SEQ ID NO: 21)

NCTKLRNATNGNDTNTTSSSRGMVGGGEMKNCSFNITTNIRGKVQKEYAL
FYKLDIAPIDNNSNNRYRLISC.

(SEQ ID NO: 22)

KCTKLKNDTNTNSSSGRMIMEKGEIDNCSFNISTSIRGKVQKEYAFFYKL
DIIPIDNDTTSYKLTSC.

(SEQ ID NO: 23)

NCTDLRNTTNTNNSTANNNSNSEGTIKGGEMKNCSFNITTSIRDKMQKEY
ALLYKLDIVSINDSTSYRLISC.

(SEQ ID NO: 24)

NCTDLGKATNTNSSNWKEEIKGEIKNCSFNITTSIRDKIQKENALFRNLD
VVPIDNASTTTNYTNYRLIHC.

### DETAILED DESCRIPTION OF THE INVENTION

Definitions and General Techniques

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics, virology and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The term “polypeptide” encompasses native or artificial proteins, protein fragments and polypeptide analogs of a protein sequence. Preferred polypeptides in accordance with the invention comprise the human heavy chain immunoglobulin molecules and the human light chain immunoglobulin molecules, as well as antibody molecules formed by combinations comprising the heavy chain immunoglobulin molecules with light chain immunoglobulin molecules, such as the κ light chain immunoglobulin molecules, as well as fragments and analogs thereof.

The term “isolated protein” or “isolated polypeptide” is a protein or polypeptide that by virtue of its origin or source of derivation (1) is not associated with naturally associated components that accompany it in its native state, (2) is free of other proteins from the same species (3) is expressed by a cell from a different species, or (4) does not occur in nature. Thus, a polypeptide that is chemically synthesized or synthesized in a cellular system different from the cell from which it naturally originates will be “isolated” from its naturally associated components. A protein or polypeptide also may be rendered substantially free of naturally associated components by isolation, using protein purification techniques well known in the art.

A protein or polypeptide is “substantially pure,” “substantially homogeneous” or “substantially purified” when at least about 60 to 75% of a sample exhibits a single species of polypeptide. The polypeptide or protein may be monomeric or multimeric. A substantially pure polypeptide or protein will typically comprise about 50%, 60, 70%, 80% or 90% W/W of a protein sample, more usually about 95%, and preferably will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

The term “polypeptide fragment” as used herein refers to a polypeptide that has an amino-terminal and/or carboxy-terminal deletion, but where the remaining amino acid sequence is identical to the corresponding positions in the naturally-occurring sequence. Fragments typically are at least 5, 6, 8 or 10 amino acids long, in certain embodiments at least 14 amino acids long, more preferably at least 20 amino acids long, usually at least 50 amino acids long, or at least 70 amino acids long.

The term “polypeptide analog” as used herein refers to a polypeptide that is comprised of a segment of at least 25 amino acids that has substantial identity to a portion of an amino acid sequence and that has at least one of the following properties: (1) specific binding to HIV-1 gp120 under suitable binding conditions or (2) ability to neutralize HIV-1. Typically, polypeptide analogs comprise a conservative amino acid substitution (or insertion or deletion) with respect to the naturally-occurring sequence. Analogs typically are at least 20 amino acids long, preferably at least 50 amino acids long or longer, and can often be as long as a full-length naturally-occurring polypeptide.

Non-peptide analogs are commonly used in the pharmaceutical industry as drugs with properties analogous to those of the template peptide. These types of non-peptide compounds are termed “peptide mimetics” or “peptidomimetics”. Fauchere, J. Adv. Drug Res. 15:29 (1986); Veber and Freidinger TINS p. 392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a desired biochemical property or pharmacological activity), such as a human antibody, but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may also be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

An “immunoglobulin” is a tetrameric molecule. In a naturally-occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as κ and λ light chains. Heavy chain constant regions are classsified as μ, Δ, γ, α, or ε, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin generally has at least two binding sites.

Immunoglobulin chains exhibit the same general structure of-relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

An “antibody” refers to an intact immunoglobulin, or to an antigen-binding portion thereof that competes with the intact antibody for specific binding. Antigen-binding portions may be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), chimeric antibodies, diabodies and polypeptides that contain at least a portion of an immunoglobulin that is sufficient to confer specific antigen binding to the polypeptide. An Fab fragment is a monovalent fragment consisting of the VL, VH, CL and CH1 domains; a F(ab′)₂ fragment is a bivalent fragment comprising two Fab fragments linked by a disulfide bridge at the hinge region; a Fd fragment consists of the VH and CH1 domains; an Fv fragment consists of the VL and VH domains of a single arm of an antibody; and a dAb fragment (Ward et al., Nature 341:544-546, 1989) consists of a VH domain. A single-chain antibody (scFv) is an antibody in which a VL and VH regions are paired to form a monovalent molecules via a synthetic linker that enables them to be made as a single protein chain (Bird et al., Science 242:423-426, 1988 and Huston et al., Proc. Natl. Acad. Sci. USA 85:5879-5883, 1988). Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., Proc. Natl. Acad. Sci. USA 90:6444-6448, 1993, and Poljak, R. J., et al., Structure 2:1121-1123, 1994). One or more CDRs may be incorporated into a molecule either covalently or noncovalently to make it an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a “bispecific” or “bifunctional” antibody has two different binding sites.

An “isolated antibody” is an antibody that (1) is not associated with naturally-associated components, including other naturally-associated antibodies, that accompany it in its native state, (2) is free of other proteins from the same species, (3) is expressed by a cell from a different species, or (4) does not occur in nature. Examples of isolated antibodies include an anti-HIV-1-gp120 antibody that has been affinity purified using a protein A or protein G column or using gp120 as an affinity ligand, an anti-HIV-1-gp120 antibody that has been synthesized by a hybridoma or other cell line in vitro, and a human anti-HIV-1-gp120 antibody derived from a transgenic mouse.

The term “human antibody” includes all antibodies that have one or more variable and constant regions derived from human immunoglobulin sequences. These antibodies may be prepared in a variety of ways, as described below.

A “humanized antibody” is an antibody that is derived from a non-human species, in which certain amino acids in the framework and constant domains of the heavy and light chains have been mutated so as to avoid or abrogate an immune response in humans. Alternatively, a humanized antibody may be produced by fusing the constant domains from a human antibody to the variable domains of a non-human species. Examples of how to make humanized antibodies may be found in U.S. Pat. Nos. 6,054,297, 5,886,152 and 5,877,293.

The term “chimeric antibody” refers to an antibody that contains one or more regions from one antibody and one or more regions from one or more other antibodies. For example, one or more of the CDRs are derived from a human anti-HIV1 antibody. Alternatively, all of the CDRs are derived from a human anti-HIV1 antibody. Alternatively, the CDRs from more than one human anti-HIV-1 antibodies, are mixed and matched in a chimeric antibody. For instance, a chimeric antibody may comprise a CDR1 from the light chain of a first human anti-HIV-1 antibody may be combined with CDR2 and CDR3 from the light chain of a second human HIV-1 antibody, and the CDRs from the heavy chain may be derived from a third anti-HIV-1 antibody. Further, the framework regions may be derived from one of the same anti-HIV-1 antibodies, from one or more different human antibodies, or from a humanized antibody.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jonsson, U., et al. (1993) Ann. Biol. Clin. 51:19-26; Jonsson, U., et al. (1991) Biotechniques 11:620-627; Johnsson, B., et al. (1995) J. Mol. Recognit. 8:125-131; and Johnnson, B., et al. (1991) Anal. Biochem. 198:268-277.

The term “K_{off}” refers to the off rate constant for dissociation of an antibody from the antibody/antigen complex.

The term “Kd” refers to the dissociation constant of a particular antibody-antigen interaction.

Fragments or analogs of antibodies or immunoglobulin molecules can be readily prepared by those of ordinary skill in the art following the teachings of this specification. Preferred amino- and carboxy-termini fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991).

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts). A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, s-N-methylarginine, and other similar amino acids and imino acids (e.g., 4-hydroxyproline). In the, polypeptide notation used herein, the lefthand direction is the amino terminal direction and the right-hand direction is the carboxy-terminal direction, in accordance with standard usage and convention.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16-3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

Unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream-sequences”; sequence regions on the DNA strand having the same sequence as the RNA and which are 3′ to the 3′ end of the RNA transcript are referred to as “downstream sequences”.

“Operably linked” sequences include both expression control sequences that are contiguous with the gene of interest and expression control sequences that act in trans or at a distance to control the gene of interest. The term “expression control sequence” as, used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. “High stringency” or “highly stringent” conditions can be used to achieve selective hybridization conditions as known in the art and discussed herein. An example of “high stringency” or “highly stringent” conditions is a method of incubating a polynucleotide with another polynucleotide, wherein one polynucleotide may be affixed to a solid surface such as a membrane, in a hybridization buffer of 6×SSPE or SSC, 50% formamide, 5×Denhardt's reagent, 0.5% SDS, 100 μg/ml denatured, fragmented salmon sperm DNA at a hybridization temperature of 42° C. for 12-16 hours, followed by twice washing at 55° C. using a wash buffer of 1×SSC, 0.5% SDS. See also Sambrook et al., supra, pp. 9.50-9.55.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is identical to all or a portion of a reference polynucleotide sequence, or that a polypeptide sequence is identical to a reference polypeptide sequence. In contrast, the term “complementary to” is used herein to mean that the complementary sequence is identical to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. U.S.A. 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 98 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, even more preferably at least 98 percent sequence identity and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

As used herein, the terms “label” or “labeled” refers to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

The term “subject” includes human and non-human subjects. A patient is a subject.

As used herein, a “linear epitope” is defined as an epitope present on an amino acid sequence that is continuous in a protein, and is identified by its presence on a synthetic peptide that is about 35 amino acids or shorter, and more preferably 20 amino acids or shorter, even more preferably, 15 amino acids or shorter.

A “disulfide-dependent epitope” is one that is destroyed by reduction of gp120 with DTT or a related reducing agent. A linear epitope may be a disulfide-dependent epitope.

Throughout this specification and claims, the word “comprise,” or variations such as “comprises” or “comprising,” will be understood to imply the inclusion of a stated integer or group of integers but not the exclusion of any other integer or group of integers.

HIV-1 env Gene

The HIV-1 env gene encodes a primary translational protein, gp160, which is proteolytically processed to two subunits, the surface subunit (SU, or gp120) or the transmembrane subunit (TM, or gp41). These subunits are believed to be noncovalently associated into heterodimers, which exist as trimeric structures in native virions. Neutralizing mabs may be directed against epitopes present on either of the HIV-1 env gene subunits. Furthermore, some such epitopes may be uniquely present on gp120-gp41 heterodimers, or on the trimeric complexes of these heterodimers. Certain neutralizing epitopes may be preferentially or exclusively exposed upon conformational rearrangements induced by binding of the gp120 to its cell surface receptors, CD4. In addition, additional epitopes may be formed upon complexing of gp120, or gp120-CD4, to one of the secondary receptors, CXCR4 or CCR5. All of these may be targets of antibodies generated by the methods described in this application, and may be used as immunogen for generating antibodies of this invention. Also, oligomeric Env complexes, such as recently described stabilized trimeric forms of HIV-1 Env proteins (Binley et al. (2000) J. Virol. 74:627-643, Yang, X. et al. (2000) J. Virol. 74:5716-5725), or native Env complexes expressed on viral particles or cell surfaces may be used as immuogen.

The HIV-1 env gene may be derived from any HIV-1 strain or clone, including strains or clones from any clade and isolate. The viruses from which these env genes were derived may by primary isolates or laboratory-adapted isolates, and the gp120s of these viruses may preferentially interact with the CXCR4 coreceptor, the CCR5 coreceptor, or may utilize a different chemokine receptor as co-receptor. In certain embodiments, gp120 is derived from a primary clade B isolate, which may be SF162, for example.

Human Antibodies and Humanization of Antibodies

Human antibodies avoid certain of the problems associated with antibodies that possess mouse or rat variable and/or constant regions. The presence of such mouse or rat derived proteins can lead to the rapid clearance of the antibodies or can lead to the generation of an immune response against the antibody by a patient. In one embodiment, the invention provides humanized anti-HIV-1-gp120 antibodies. In another embodiment, the invention provides fully human anti-HIV-1-gp120 antibodies through the immunization of a rodent in which human immunoglobulin genes have been introduced so that the rodent produces fully human antibodies. Fully human antibodies are expected to minimize the immunogenic and allergic responses intrinsic to mouse or mouse-derivatized Mabs and thus to increase the efficacy and safety of the administered antibodies. The use of fully human antibodies can be expected to provide a substantial advantage in the treatment of various human diseases, such as an HIV-1 infection, which may require repeated antibody administrations.

Methods of Producing Antibodies and Antibody-Producing Cell Lines

Immunization

In one embodiment of the instant invention, human antibodies are produced by immunizing a non-human animal, some of whose cells comprise all or a functional portion of the human immunoglobulin heavy and/or light chain loci, with, inter alia, a gp120 antigen, a gp41 antigen, gp120-gp41 heterodimers, trimeric complexes of these heterodimers, or any antigen comprising gp120 and/or gp41 and other host cellular receptor proteins. In a preferred embodiment, the non-human transgenic animal has the ability to make human antibodies but is deficient in the ability to make its cognate antibodies. In preferred embodiments, the non-human animal is a mammal. In a more preferred embodiment, the non-human animal is a mouse. In an even more preferred embodiment, the non-human animal is a XENOMOUSE® animal.

XENOMOUSE® animals are any one of a number of engineered mouse strains that comprise large fragments of the human immunoglobulin loci (generally comprises some or all of the human heavy and light chain loci) and is deficient in mouse antibody production. See, e.g., Green et al. Nature Genetics 7:13-21 (1994) and U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000.

Early XENOMOUSE® animal strains were engineered with yeast artificial chromosomes (YACs) containing 245 kb and 190 kb-sized germline configuration fragments of a human heavy chain locus and a kappa light chain locus, respectively, which contained core variable and constant region sequences. Id. Subsequent XENOMOUSE® animals contain approximately 80% of the human antibody repertoire through introduction of megabase sized, germline configuration YAC fragments of the human heavy chain loci and kappa light chain loci. See Mendez et al. Nature Genetics 15:146-156 (1997), Green and Jakobovits J. Exp. Med. 188:483-495 (1998), and U.S. patent application Ser. No. 08/759,620, filed Dec. 3, 1996, the disclosures of which are hereby incorporated by reference. XENOMOUSE® animals produce an adult-like human repertoire of fully human antibodies, and generates antigen-specific human antibodies.

In another embodiment, the non-human animal comprising human immunoglobulin gene loci are animals that have a “minilocus” of human immunoglobulins. In the minilocus approach, an exogenous Ig locus is mimicked through the inclusion of individual-genes from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described, inter alia, in U.S. Pat. Nos. 5,545,807, 5,545,806, 5,625,825, 5,625,126, 5,633,425, 5,661,016, 5,770,429, 5,789,650, 5,814,318, 5,591,669, 5,612,205, 5,721,367, 5,789,215, and 5,643,763, hereby incorporated by reference.

An advantage of the minilocus approach is the rapidity with which constructs including portions of the Ig locus can be generated and introduced into animals. However, a potential disadvantage of the minilocus approach is that there may not be sufficient immunoglobulin diversity to support full B-cell development, such that there may be lower antibody production.

In another embodiment, the invention provides a method for making anti-HIV-1-gp120 antibodies from non-human, non-mouse animals by immunizing non-human transgenic animals that comprise human immunoglobulin loci. One may produce such animals using the methods described in U.S. Pat. Nos. 5,916,771, 5,939,598, 5,985,615, 5,998,209, 6,075,181, 6,091,001, 6,114,598 and 6,130,364. See also WO 91/10741, published Jul. 25, 1991, WO 94/02602, published Feb. 3, 1994, WO 96/34096 and WO 96/33735, both published Oct. 31, 1996, WO 98/16654, published Apr. 23, 1998, WO 98/24893, published Jun. 11, 1998, WO 98/50433, published Nov. 12, 1998, WO 99/45031, published Sep. 10, 1999, WO 99/53049, published Oct. 21, 1999, WO 00 09560, published Feb. 24, 2000 and WO 00/037504, published Jun. 29, 2000. The methods disclosed in these patents may modified as described in U.S. Pat. No. 5,994,619. In a preferred embodiment, the non-human animals may be rats, sheep, pigs, goats, cattle or horses.

In another embodiment, the invention provides a method for making anti-HIV-1 gp120 antibodies from non-human, non-transgenic animals. In this embodiment, the non-human, non-transgenic animals are immunized with an antigen as described below and antibodies are produced by these animals. Antibody-producing cells may be isolated from these animals, immortalized by any means known in the art, for example, preferably by fusion with myelomas to produce hybridomas, and subsequently engineered to produce “humanized antibodies” such that they do not cause an immune response in a human using techniques known to those of skill in the art and as described further below.

Human Monoclonal Antibodies Against HIV-1 gp120

As shown in Example 1, the ability to hyperimmunize XENOMOUSE® mice with preselected immunogens and under optimized immunization protocols allowed the isolation of large numbers of antibodies against multiple epitopes present in the target gp120 antigen, thus improving the ability to saturate the target antigen.

This strategy produced neutralizing antibodies that are rare or absent in clinical samples currently used as the source of human Mabs. As an example, only a minority of humans produce antibodies against conserved V1/V2 epitopes (see Kayman, S. C. et al. (1994) J. Virol. 68:400-410), perhaps due to the relatively poor immunogenicity of these regions or the inappropriate presentation of these epitopes during viral infection and propagation of clinical strains of virus. In contrast to this, XENOMOUSE® animals immunized with recombinant gp120 (“rgp120”) produced relatively high titers of antibodies against V1/V2 epitopes.

The availability of mutant and deglycosylated rgp120s and variable domain fusion proteins may further improve immunogenicity of epitopes that may be secluded or poorly immunogenic in native proteins and virions. Furthermore, the use of native viral Envelope proteins expressed on the surface of cells or virions in the natural oligomeric form both as immunogens and in screening assays may allow identification of unstable or metastable epitopes that are not well-represented or not represented at all on purified soluble antigens.

The availability of an efficient functional screen to select hybridomas producing Mabs with HIV neutralizing activities may allow the isolation of antibodies targeted against native epitopes that may not be expressed on available purified antigens. These may include highly conformational epitopes, epitopes dependent on oligomeric complexes, or epitopes located on the TM protein or on Env-receptor complexes. The specificity of such assays may allow more efficient screening assays, since irrelevant antibodies (i.e., those against non-neutralizing sites) can be bypassed, thereby facilitating analyses of larger number of fusions than currently feasible.

To produce an anti-HIV-1-gp120 antibody, a non-human transgenic animal comprising some or all of the human immunoglobulin loci is immunized with an HIV-1 gp120 antigen or a fragment thereof. In a preferred embodiment, the non-human animal has the ability to produce human antibodies but is deficient in producing its cognate antibodies. In a more preferred embodiment, the non-human animal is a XENOMOUSE® animal.

Human monoclonal antibodies with potent neutralizing activity against multiple primary HIV-1 isolates are generated by immunizing XENOMOUSE® mice with various forms of HIV-1 env antigens. These antigens may be recombinant gp120, gp160 or gp41, portions thereof, or fusion proteins comprising gp120, gp160 or gp41 or portions thereof. Furthermore, some epitopes may be uniquely present on gp120-gp41 heterodimers, or on the trimeric complexes of these heterodimers. Certain neutralizing epitopes may be preferentially or exclusively exposed upon conformational rearrangements induced by binding of the gp120 to its cell surface receptors, CD4. In addition, additional epitopes may be formed upon complexing of gp120, or gp120-CD4, to one of the secondary receptors, CXCR4 or CCR5. All of these may be targets of antibodies generated by the methods described in this application, and may be used as immunogen for generating antibodies of this invention. Also, oligomeric Env complexes, such as recently described stabilized trimeric forms of HIV-1 Env proteins (Binley et al. (2000) J. Virol. 74:627-643, Yang, X. et al. (2000) J. Virol. 74:5716-5725), or native Env complexes expressed on viral particles or cell surfaces may be used as immuogen. Immunogens include recombinant antigens derived from both clade B and non-clade B strains, including both CXCR4 (X4)- and CCR5 (R5)-tropic isolates. In a preferred embodiment, the HIV-1 gp120 is a recombinant gp120 (rgp120). In another preferred embodiment, the antigens are derived from a primary isolate of HIV-1. In a more preferred embodiment, the immunogen, such as a rgp120, is derived from SF162 isolate of HIV-1.

Immunizations are also performed with intact whole viruses, including, but not limited to, live-attenuated HIV-1, inactivated HIV-1, or chimeric viruses that display HIV-1 env complexes on their surfaces, for example, heterologous Simian:Human Immunodeficiency Virus (SHIV), heterologous Murine:Human Immunodeficiency Virus, Vaccinia:HIV-1 chimeras, or Picornaviruses (e.g., Poliovirus, Human Rhinovirus) displaying HIV-1 gp120 epitopes on their surfaces. In a preferred embodiment, such whole-virus immunogens act as protein antigens that are not replication-competent (e.g., inactivated HIV-1, SHIV). In a more preferred embodiment, such whole-virus immunogens will be replication-competent in mice (e.g., Murine:Human Immunodeficiency Virus, or another murine virus displaying HIV-1 gp120 immunogens.

Immunizations are also performed with native env complexes displayed in native or alternative environments. Such native or alternative approaches include, but are not limited to, intact and stabilized viral particles (e.g., ghost cells, liposomes, or beads displaying native HIV-1 env complexes on their surfaces) or mouse cells transfected with complete HIV-1 env genes.

In another embodiment, immunizations are performed with DNA that encodes HIV-1 immunogens, such as gp120 immunogens.

Hybridoma screening are performed both by standard binding assays with appropriate antigens, including viral particles, and by direct functional screening assays, using an ultra-sensitive luciferase-based HIV-neutralization assay.

Antibodies isolated in initial screening assays are fully characterized for epitope specificity, strain distribution and neutralizing potency against a panel of viral isolates. Epitope characterizations utilize binding assays to various peptides and recombinant miniproteins corresponding to specific domains of env proteins, and a panel of viral gp120s, including proteins with deletions of specific domains. Gp120-binding competition assays are performed with soluble CD4 (sCD4) or Mabs against well-characterized epitopes, using both ELISA and Biacore methods. Neutralizing assays are performed with a broad range of viral isolates, including T cell-tropic and M-tropic primary isolates, including both clade B and foreign clade isolates, using both PBMC and cell line-based assays. Neutralization activity of the antibodies of this invention can be measured in several different ways. The most useful assay is a single cycle infectivity assay, using the NL4-3 luciferase virus, pseudotyped with HIV-1 env. The NL4-3 luc virus has a defective env gene, and has the luc gene in place of nef. See Chen, B. K. et al. (1994) J. Virol. 68:654-660. When complemented in trans with a functional env gene, the resulting virions transduce luc activity upon entry into susceptible cells. This assay is quite rapid, quantitative, and sensitive. Luciferase activity can be measured quickly and accurately as early as two days after infection, using a 96-well plate fluorometer, and the assay has a very large dynamic range. Those antibodies that neutralize HIV-1 in vitro could neutralize HIV-1 in viva. The fact that these antibodies neutralize HIV-1 in vivo may be further confirmed in animal model systems, such as in hu-PBL-SCID mice (Safrit (1993) AIDS 7:15-21) or neonatal macaques (Hofmann-Lehmann (2001) J. Virol. 75:7470-7480).

Example 1 provides a protocol for immunizing a XENOMOUSE® animal with full-length recombinant gp120 of the SF162 primary isolate of HIV-1 and provides antibodies that bind HIV-1 gp120 and that neutralize HIV-1.

In one embodiment of this invention, an isolated human antibody or antigen-binding portion thereof that specifically binds to HIV-1 gp120 protein (such as HIV-1_{SF162} gp120 protein) and that has HIV-1 neutralizing activity is provided, wherein said antibody or antigen-binding portion thereof recognizes an epitope (preferably a linear epitope) on a V1/V2 domain of HIV-1 gp120, wherein said epitope is dependent on the presence of a sequence in the V1 loop. In a preferred embodiment, said antibody described in this paragraph or antigen-binding portion thereof does not bind an HIV-1 strain Case-A2 V1/V2 domain specific epitope. In yet another preferred embodiment, said antibody described in this paragraph or antigen-binding portion thereof does not bind the V1/V2 domain of the gp120 of HIV-1 strain Case A2. In a more preferred embodiment, said antibody described in this paragraph or antigen-binding portion thereof has HIV-1_{SF162} neutralizing activity. In another more preferred embodiment, said antibody described in this paragraph or antigen-binding portion thereof recognizes a linear epitope on a V1 domain of HIV-1_{SF162} gp120. In an even more preferred embodiment, said antibody described in this paragraph or antigen-binding portion thereof recognizes a linear epitope on a V1 domain of HIV-1_{SF162} gp120 and the antibody or antigen binding portion thereof has HIV-1_{SF162} neutralizing activity. In another even more preferred embodiment, said antibody described in this paragraph or antigen-binding portion thereof has HIV-1_{SF162} neutralizing activity and that SF162 neutralizing activity is approximately as strong as the HIV-1_{SF162} neutralizing activity of human monoclonal antibody selected from the group consisting of 45D1/B7, secreted by a hybridoma designated by ATCC Accession Number PTA-3002, 58E1/B3, secreted by a hybridoma designated by ATCC Accession Number PTA-3003 and 64B9/A6, secreted by a hybridoma designated by ATCC Accession Number PTA-3004. As shown in FIG. 9 and Example 1, Mab 45D1/B7 neutralized HIV-1_{SF162} virus with an ND50 of about 1.9 μg/ml; Mab 58E1/B3 neutralized HIV-1_{SF162} virus with an ND50 of about 0.55 μg/ml; and Mab 64B9/A6 neutralized HIV-1_{SF162} virus with an ND50 of about 0.29 μg/ml. In another preferred embodiment, said antibody described in this paragraph or antigen-binding portion thereof described in this paragraph specifically binds to a peptide consisting of SEQ ID NO: 3. In a more preferred embodiment, said antibody described in this paragraph or antigen-binding-portion thereof specifically binds to a peptide consisting of SEQ ID NO: 3, and does not specifically bind to a peptide consisting of SEQ ID NO: 2. In an even more preferred embodiment, said antibody described in this paragraph or antigen-binding portion thereof is a human monoclonal antibody (human Mab). In an even more preferred embodiment, said human Mab described above is selected from the group consisting of 35D10/D2, secreted by a hybridoma designated by ATCC Accession Number PTA-3001, 40H2/C7, secreted by a hybridoma designated by ATCC Accession Number PTA-3006, 43A3/E4, secreted by a hybridoma designated by ATCC Accession Number PTA-3005, 43C7/B9, secreted by a hybridoma designated by ATCC Accession Number PTA-3007, 45D1/B7, secreted by a hybridoma designated by ATCC Accession Number PTA-3002, 46E3/E6, secreted by a hybridoma designated by ATCC Accession Number PTA-3008, 58E1/B3, secreted by a hybridoma designated by ATCC Accession Number PTA-3003, and 64B9/A6, secreted by a hybridoma designated by ATCC Accession Number PTA-3004. Mabs 35D10/D2, 40H2/C7, 43A3/E4, 43C7/B9, 45D1/B7, 46E3/E6, 58E1/B3 and 64B9/A6 neutralized HIV-1_{SF162}, many with quite potent end points (FIG. 9). All eight of these antibodies were specific for linear V1 epitopes.

In another embodiment, an isolated human antibody or antigen-binding portion thereof that specifically binds to HIV-1 gp120 protein (such as HIV-1_{SF162} gp120 protein) and that has HIV-1 neutralizing activity is provided, wherein said antibody or antigen-binding portion thereof recognizes an epitope (preferably a linear epitope) on a V1/V2 domain of HIV-1 gp120, such as HIV-1_{SF162} gp120, wherein said epitope is dependent on the presence of a sequence in the V2 domain. In a more preferred embodiment, said antibody described in this paragraph or antigen-binding portion thereof recognizes an epitope (preferably a linear epitope) on a V2 domain of HIV-1 gp120, such as HIV-1_{SF162} gp120. In another preferred embodiment, said antibody described in this paragraph or antigen-binding portion thereof has HIV-1 neutralizing activity. In a more preferred embodiment, said antibody described in this paragraph or antigen-binding portion thereof has HIV-1_{SF162} neutralizing activity. In another preferred embodiment, said antibody described in this paragraph or antigen-binding portion thereof recognizes a linear epitope on a V2 domain of HIV-1 gp120, such as HIV-1_{SF162} gp120, and the antibody or antigen binding portion thereof has HIV-1_{SF162} neutralizing activity. In a preferred embodiment, said antibody described in this paragraph or antigen-binding portion thereof specifically binds to at least three R5 clade HIV-1 gp120 proteins. In a preferred embodiment, said antibody described in this paragraph or antigen-binding portion thereof specifically binds to a peptide consisting of SEQ ID NO: 4. In another preferred embodiment, said antibody described in this paragraph or antigen-binding portion thereof does not specifically bind to a gp120 of HIV-1 IIIB, or related clones, such as HXB2, HXB2d and BH10. In a more preferred embodiment, said human antibody described in this paragraph or antigen-binding portion thereof is a human monoclonal antibody. In an even more preferred embodiment, said human Mab is Mab 8.22.2, secreted by a hybridoma designated by ATCC Accession Number ______.

In another embodiment of this invention, an isolated human monoclonal antibody or antigen-binding portion thereof that specifically binds to an epitope on a V3 region of HIV-1 gp120 is provided, wherein, preferably, said antibody binds to an epitope in the V3 region of HIV-1_{SF162} gp120, and wherein said antibody does not specifically bind to a peptide consisting of SEQ ID NO:9 (V3 amino acids 1-20 of gp120 of HIV-1 MN strain). In a more preferred embodiment, said antibody described in this paragraph or antigen-binding portion thereof specifically binds to a HIV-1 gp120 protein (such as HIV-1_{SF162} gp120 protein). In a more preferred embodiment, said antibody described in this paragraph or antigen-binding portion thereof binds to an epitope (linear or conformational) on the V3 region of HIV-1_{SF162} gp120. In another preferred embodiment, said antibody described in this paragraph or antigen-binding portion thereof has HIV-1 neutralizing activity. In a more preferred embodiment, said antibody described in this paragraph or antigen-binding portion thereof has HIV-1_{SF162} neutralizing activity. In an even more preferred embodiment, said antibody described in this paragraph or antigen-binding portion thereof is human monoclonal antibody 8.27.3, secreted by a hybridoma designated by ATCC Accession Number PTA-3009 or Mab 8E11/A8, secreted by hybridoma designated by ATCC Accession Number ______. As shown in Example 1, Mab 8.27.3 and mab 8E11/A8 did not specifically bind MN V3 1-20 (SEQ ID NO: 9). As shown in FIG. 9, Mab 8.27.3 was shown to have a SF162 HIV-1 virus neutralizing activity of about 0.11 μg/ml and Mab 8E11/A8 was shown to have a SF162 HIV-1 virus neutralizing, activity of about 2.6 μg/ml. As shown in FIG. 2 and Example 1, Mabs .694 and 447-52D (described in U.S. Pat. No. 5,914,109), included here for comparison purpose, specifically bound to MN V3 1-20 (SEQ ID NO: 9). In contrast, human monoclonal antibodies 8.27.3 and 8E11/A8, made according to the above-identified procedure (see also Example 1), did not specifically bind MN V3 1-20 (SEQ ID NO: 9) or MN V3 21-40 (SEQ ID NO: 11), but did bind to a larger peptide containing all 33 amino acids of the MN V3 loop (TRPNYNKRKRIHIGPGRAFYTTKNIIGTIRQAH) (SEQ ID NO: 7). Mab 8.27.3 did not bind MN V3 11-30 (SEQ ID NO: 10), whereas Mab 8E11/A8 did.

In a more preferred embodiment, the antibody of this invention or antigen-binding portion thereof has HIV-1 neutralizing activity for more than one primary isolate of HIV-1. In some embodiments, the antibody of this invention or antigen-binding portion thereof has HIV-1 neutralizing activity for only one primary isolate of HIV-1. In more preferred embodiments, the antibody of this invention or antigen-binding portion thereof has HIV-1 neutralizing activity for more than one primary isolate of HIV-1 from members of more than one clade. In another even more preferred embodiment, the antibody of this invention or antigen-binding portion thereof has HIV-1 neutralizing activity in vivo. The fact that these antibodies neutralize HIV-1 in vivo may be further confirmed in animal model systems, such as in hu-PBL-SCID mice (Safrit (1993) AIDS 7:15-21) or neonatal macaques (Hofmann-Lehmann (2001) J. Virol. 75:7470-7480).

This invention provides an isolated human antibody. Said antibody may be a human monoclonal antibody.

An antibody of this invention, or portion thereof, can inhibit the binding of HIV-1 gp120 to human CXCR4 receptor. Any conventional assays known in the art, either in vitro or in vivo, may be used to measure such inhibition.

An antibody of this invention, or portion thereof, can inhibit the binding of HIV-1 gp120 to human CCR5 receptor. Any conventional assays known in the art, either in vitro or in vivo, may be used to measure such inhibition.

Production of Antibodies and Antibody-Producing Cell Lines

Immunization

Immunization of animals may be done by any method known in the art. See, e.g., Harlow and Lane, Antibodies: A Laboratory Manual, New York: Cold Spring Harbor Press, 1990. Methods for immunizing non-human animals such as mice, rats, sheep, goats, pigs, cattle and horses are well known in the art. See, e.g., Harlow and Lane and U.S. Pat. No. 5,994,619. In a preferred embodiment, the antigen is administered with or without an adjuvant to stimulate the immune response. Such adjuvants include, inter alia, complete or incomplete Freund's adjuvant, RIBI (muramyl dipeptides) or ISCOM (immunostimulating complexes). Such adjuvants may protect the polypeptide from rapid dispersal by sequestering it in a local deposit, or they may contain substances that stimulate the host to secrete factors that are chemotactic for macrophages and other components of the immune system. Preferably, if a polypeptide is being administered, the immunization schedule will involve two or more administrations of the polypeptide, spread out over several weeks.

After immunization of an animal with an antigen, antibodies and/or antibody-producing cells may be obtained from the animal. In one embodiment, antibody-containing serum is obtained from the animal by bleeding or sacrificing the animal. The serum may be used as it is obtained from the animal, an immunoglobulin fraction may be obtained from the serum, or the antibodies may be purified from the serum. It is well known to one of ordinary skill in the art that serum or immunoglobulins obtained in this manner will be polyclonal. The disadvantage is using polyclonal antibodies prepared from serum is that the amount of antibodies that can be obtained is limited and the polyclonal antibody has a heterogeneous array of properties.

In another embodiment, antibody-producing cells may be immortalized by, e.g., Epstein-Barr virus, by fusion with suitable immortal myeloma cell lines, or by any other conventional methods known in the art.

In a preferred embodiment, antibody-producing immortalized hybridomas may be prepared from the immunized animal. After immunization, the animal is sacrificed-and the splenic B cells are fused to immortalized myeloma cells as is well-known in the art. See, e.g., Harlow and Lane, supra. In a preferred embodiment, the myeloma cells do not secrete immunoglobulin polypeptides (a non-secretory cell line). After fusion and antibiotic selection, the hybridomas are screened using, for example, HIV-1 gp120, or a portion of HIV-1 gp120, or a cell expressing HIV-1 gp120. In a preferred embodiment, the initial screening is performed using, for example, an enzyme-linked immunoassay (ELISA) or a radioimmunoassay. In a more preferred embodiment, an ELISA is used for initial screening. An example of ELISA screening is provided in WO 00/37504, herein incorporated by reference.

Antibody-producing hyridomas are selected, cloned and further screened for desirable characteristics, including robust hybridoma growth, high antibody production and desirable antibody characteristics, as discussed further below. Hybridomas may be expanded in vivo in syngeneic animals, in animals that lack an immune system, e.g., nude mice, or in cell culture in vitro. Methods of selecting, cloning and expanding hybridomas are well known to those of ordinary skill in the art.

In a preferred embodiment, the immunized animal is a non-human animal that expresses human immunoglobulin genes and the splenic B cells are fused to a myeloma derived from the same species as the non-human animal. In a more preferred embodiment, the immunized animal is a XENOMOUSE® animal and the myeloma cell line is a non-secretory mouse myeloma.

In one embodiment, hybridomas are produced that produce human anti-HIV-1-gp120 antibodies. In a preferred embodiment, the hybridomas are mouse hybridomas, as described above. In another preferred embodiment, the hybridomas are produced in a non-human, non-mouse species such as rats, sheep, pigs, goats, cattle or horses. In another embodiment, the hybridomas are human hybridomas, in which a human non-secretory myeloma is fused with a human cell expressing an anti-HIV-1-gp120 antibody.

In another embodiment, antibody-producing cells may be prepared from a human who has an HIV-1 infection and who expresses anti-HIV-1-gp120 antibodies. Cells expressing the anti-HIV-1-gp120 antibodies may be isolated by isolating white blood cells and subjecting them to fluorescence-activated cell sorting (FACS) or by panning on plates coated with HIV-1 gp120 or a portion thereof. These cells may be fused with a human non-secretory myeloma to produce human hybridomas expressing human anti-HIV-1-gp120 antibodies.

Nucleic Acids, Vectors, Host Cells and Recombinant Methods of Making Antibodies

The nucleic acid molecule encoding either the entire heavy and light chains of an anti-HIV-1-gp120 antibody or the variable regions thereof may be obtained from any source that produces such an antibody.

In one embodiment of the invention, the nucleic acid molecules may be obtained from a hybridoma that expresses an antibody, such as from one of the hybridomas described above. Methods of isolating mRNA encoding an antibody are well-known in the art. See, e.g., Sambrook et al., supra. The mRNA may be used to produce cDNA for use in the polymerase chain reaction (PCR) or cDNA cloning of antibody genes. In a preferred embodiment, the nucleic acid molecule is derived from a hybridoma that has as one of its fusion partners a transgenic non-human animal cell that expresses human immunoglobulin genes. In an even more preferred embodiment, the fusion partner animal cell is derived from a XENOMOUSE® animal. In another embodiment, the hybridoma is derived from a non-human, non-mouse transgenic animal as described above. In another embodiment, the hybridoma is derived from a non-human, non-transgenic animal. The nucleic acid molecules derived from a non-human, non-transgenic animal may be used, e.g., for humanized antibodies.

In a preferred embodiment, the heavy chain of an anti-HIV-1-gp120 antibody may be constructed by fusing a nucleic acid molecule encoding the variable domain of a heavy chain with a constant domain of a heavy chain. Similarly, the light chain of an anti-HIV-1-gp120 may be constructed by fusing a nucleic acid molecule encoding the variable domain of a light chain with a constant domain of a light chain.

In another embodiment, an anti-HIV-1-gp120 antibody-producing cell itself may be purified from a non-human, non-mouse animal. In one embodiment, the antibody-producing cell may be derived from a transgenic animal that expresses human immunoglobulin genes and has been immunized with a suitable antigen. The transgenic animal may be a mouse, such as a XENOMOUSE® animal, or another non-human transgenic animal. In another embodiment, the anti-HIV-1-gp120 antibody-producing cell is derived from a non-transgenic animal. In another embodiment, the anti-HIV-1-gp120 antibody-producing cell may be derived from a human patient with an HIV-1 infection who produces anti-HIV-1-gp120 antibodies. The mRNA from the antibody-producing cells may be isolated by standard techniques, amplified using PCR and screened using standard techniques to obtain nucleic acid molecules encoding anti-HIV-1 gp120 heavy and light chains.

In another embodiment, the nucleic acid molecules may be used to make vectors using methods known to those having ordinary skill in the art. See, e.g., Sambrook et al., supra, and Ausubel et al., supra. In one embodiment, the vectors may be plasmid or cosmid vectors. In another embodiment, the vectors may be viral vectors. Viral vectors include, without limitation, adenovirus, retrovirus, adeno-associated viruses and other picorna viruses, hepatitis virus and baculovirus. The vectors may also be bacteriophage including, without limitation, M13.

The nucleic acid molecules may be used to recombinantly express large quantities of antibodies, as described below. The nucleic acid molecules may also be used to produce chimeric antibodies, single chain antibodies, immunoadhesins, diabodies, mutated antibodies (such as antibodies with greater binding, affinity for the antigen) and antibody derivatives, as described further below. If the nucleic acid molecules are derived from a non-human, non-transgenic animal, the nucleic acid molecules may be used for antibody humanization, also as described below.

In one embodiment, the nucleic acid molecules encoding the variable region of the heavy (VH) and light (VL) chains are converted to full-length antibody genes. In one embodiment, the nucleic acid molecules encoding the VH and VL chain are converted to full-length antibody genes by inserting them into expression vectors already encoding heavy chain constant and light chain constant regions, respectively, such that the VH segment is operatively linked to the CH segment(s) within the vector and the VI, segment is operatively linked to the CL segment within the vector. In another embodiment, the nucleic acid molecules encoding the VH and/or VL chains are converted into full-length antibody genes by linking the nucleic acid molecule encoding a VH chain to a nucleic acid molecule encoding a CH chain using standard molecular biological techniques. The same may be achieved using nucleic acid molecules encoding VL and CL chains. The sequences of human heavy and light chain constant region genes are known in the art. See, e.g., Kabat et al., Sequences of Proteins of Immunological Interest, 5th Ed., NIH Publ. No. 91-3242, 1991. The CDR1, CDR2 and CDR3 regions of the heavy chain of an antibody-may also be determined. Id.

In another embodiment, the nucleic acid molecules of the invention may be used as probes or PCR primers for specific antibody sequences. For instance, a nucleic acid molecule probe may be used in diagnostic, methods or a nucleic acid molecule PCR primer may be used to amplify-regions of DNA that could be used, inter alia, to isolate nucleic acid sequences for use in producing variable domains of the antibodies of the present invention. In a preferred embodiment, the nucleic acid molecules are oligonucleotides. In a more preferred embodiment, the oligonucleotides are from highly variable regions of the heavy and light chains of the antibody of interest. In an even more preferred embodiment, the oligonucleotides encode all or a part of one or more of the CDRs.

The above-described methods can be used to produce an antibody comprising the heavy chain, heavy and light chain or the CDR1, CDR2 and CDR3 of any one of the antibodies of this invention.

Vectors

To express the antibodies, or antibody portions of the invention, DNAs encoding partial or full-length light and heavy chains, obtained as described above, are inserted into expression vectors such that the genes are operatively linked to transcriptional and translational control sequences. Expression vectors include plasmids, retroviruses, cosmids, YACs, EBV derived episomes, and the like. The antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector. In a preferred embodiment, both genes are inserted into the same expression vector. The antibody genes are inserted into the expression vector by standard methods (e.g., ligation of complementary restriction sites on the antibody gene fragment and vector, or blunt end ligation if no restriction sites are present).

A convenient vector is one that encodes a functionally complete human C_{H} or C_{L} immunoglobulin sequence, with appropriate restriction sites engineered so that any V_{H} or V_{L} sequence can be easily inserted and expressed, as described above. In such vectors, splicing usually occurs between the splice donor site in the inserted J region and the splice acceptor site preceding the human C region, and also at the splice regions that occur within the human C_{H} exons. Polyadenylation and transcription termination occur at native chromosomal sites downstream of the coding regions. The recombinant expression vector can also encode a signal peptide that facilitates secretion of the antibody chain from a host cell. The antibody chain gene may be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors of the invention may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

Non-Hybridoma Host Cells and Methods of Recombinantly Producing Protein

Nucleic acid molecules encoding anti-HIV-1-gp120 antibodies and vectors comprising these antibodies can be used for transformation of a suitable mammalian host cell. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, and direct microinjection of the DNA into nuclei. In addition, nucleic acid molecules may be introduced into mammalian cells by viral vectors. Methods of transforming cells are well known in the art. See, e.g., U.S. Pat. Nos. 4,399,216, 4,912,040, 4,740,461, and 4,959,455 (the disclosures of which are hereby incorporated herein by reference).

Mammalian cell lines available as hosts for expression are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human-hepatocellular carcinoma cells (e.g., Hep G2), A549 cells, and a number of other cell lines. Cell lines of particular preference are selected through determining which cell lines have high expression levels. Other cell lines that may be used are insect cell lines, such as Sf9 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

Transgenic Animals

Antibodies of the invention can also be produced transgenically through the generation of a mammal or plant that is transgenic for genes encoding the immunoglobulin heavy and light chain sequences of the antibody of interest and production of the antibody in a recoverable form therefrom. In connection with the transgenic production in mammals, antibodies can be produced in, and recovered from, the milk of goats, cows, or other mammals. See, e.g., U.S. Pat. Nos. 5,827,690, 5,756,687, 5,750,172, and 5,741,957.

In another embodiment, the transgenic animals or plants comprise nucleic acid molecules encoding anti-HIV-1-gp120 antibodies. In a preferred embodiment, the transgenic animals or plants comprise nucleic acid molecules encoding heavy and light chains specific for HIV-1 gp120.

In another embodiment, the transgenic animals or plants comprise nucleic acid molecules encoding a modified antibody such as a single-chain antibody, a chimeric antibody or a humanized antibody. The anti-HIV-1-gp120 antibodies may be made in any transgenic animal or plants. In a preferred embodiment, the non-human animals are, without limitation, mice, rats, sheep, pigs, goats, cattle or horses; and the plants are, without limitation, tobacco, corn, or soy. As will be appreciated, proteins may also be generated in eggs that are transgenic for the genes encoding the proteins, such as chicken eggs, among other things.

Phage Display Libraries

Recombinant anti-HIV-1-gp120 antibodies of the invention in addition to the anti-HIV-1-gp120 antibodies disclosed herein can be isolated by screening of a recombinant combinatorial antibody library, preferably a scFv phage display library, prepared using human V_{L} and V_{H} cDNAs prepared from mRNA derived from human lymphocytes. Methodologies for preparing and screening such libraries are known in the art. There are commercially available kits for generating phage display libraries (e.g., the Pharmacia Recombinant Phage Antibody System, catalog no. 27-9400-01; and the Stratagene SurfZAP™ phage display kit, catalog no. 240612). There are also other methods and reagents that can be used in generating and screening antibody display libraries (see. e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum. Antibod. Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc Acid Res 19:4133-4137; and Barbas et al. (1991) Proc. Natl. Acad. Sci. USA 88:7978-7982.

In a preferred embodiment, to isolate human anti-HIV-1-gp120 antibodies with the desired characteristics, a human anti-HIV-1-gp120 antibody as described herein is first used to select human heavy and/or light chain sequences having similar binding activity toward HIV-1 gp120 respectively, using the epitope imprinting methods described in Hoogenboom et al., PCT Publication No. WO 93/06213. The antibody libraries used in this method are preferably scFv libraries prepared and screened as described in McCafferty et al., PCT Publication No. WO 92/01047, McCafferty et al., Nature (1990) 348:552-554; and Griffiths et al., (1993) EMBO J 12:725-734. The scFv antibody libraries preferably are screened using HIV-1 gp120 as the antigen, respectively.

Once initial human V_{L} and V_{H} segments are selected, “mix and match” experiments, in which different pairs of the initially selected V_{L} and V_{H} segments are screened for HIV-1 gp120 binding, are performed to select preferred VL/VH pair combinations. Additionally, to further improve the quality of the antibody, the VL and VH segments of the preferred VL/VH pair(s) can be randomly mutated, preferably within the CDR3 region of VH and/or VL, in a process analogous to the in vivo somatic mutation process responsible for affinity maturation of antibodies during a natural immune response. This in vitro affinity maturation can be accomplished by amplifying VH and VL regions using PCR primers complimentary to the VH CDR3 or VL CDR3, respectively, which primers have been “spiked” with a random mixture of the four nucleotide bases at certain positions such that the resultant PCR products encode VH and VL segments into which random mutations have been introduced into the VH and/or VL CDR3 regions. These randomly mutated VH and VL segments can be rescreened for binding to the antigen.

Following screening and isolation of an antibody of the invention from a recombinant immunoglobulin display library, nucleic acid encoding the selected antibody can be recovered from the display package (e.g., from the phage genome) and subcloned into other expression vectors by standard recombinant DNA techniques. If desired, the nucleic acid can be further manipulated to create other antibody forms of the invention, as described below. To express a recombinant human antibody isolated by screening of a combinatorial library, the DNA encoding the antibody is cloned into a recombinant expression vector and introduced into a mammalian host cells, as described above.

Class Switching

Another aspect of the instant invention is to provide a mechanism by which the class of an antibody of this invention may be switched with another. In one aspect of the invention, a nucleic acid molecule encoding VL or VH is isolated using methods well-known in the art such that it does not include any nucleic acid sequences encoding CL or CH. The nucleic acid molecule encoding VL or VH are then operatively linked to a nucleic acid sequence encoding a CL or CH from a different class of immunoglobulin molecule. This may be achieved using a vector or nucleic acid molecule that comprises a CL or CH chain, as described above. For example, an antibody that was originally IgM may be class switched to an IgG. Further, the class switching may be used to convert one IgG subclass to another, e.g., from IgG1 to IgG2.

Antibody Derivatives

One may use the nucleic acid molecules described above to generate antibody derivatives using techniques and methods known to one of ordinary skill in the art.

Humanized Antibodies

As was discussed above in connection with human antibody generation, there are advantages to producing antibodies with reduced immunogenicity. This can be accomplished to some extent using techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit. Reviews in Immunol. 12125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085).

Mutated Antibodies

In another embodiment, the nucleic acid molecules, vectors and host cells may be used to make mutated antibodies. The antibodies may be mutated in the variable domains of the heavy and/or light chains to alter a binding property of the antibody. For example, a mutation may be made in one or more of the CDR regions to increase or decrease the K_{d} of the antibody for its antigen, to increase or decrease K_{off}, or to alter the binding specificity of the antibody. Techniques in site-directed mutagenesis are well-known in the art. See, e.g., Sambrook et al. and Ausubel et al., supra. In a preferred embodiment, mutations are made at an amino acid residue that is known to be changed compared to germline in a variable region of an antibody of the present invention. In another embodiment, the nucleic acid molecules are mutated in one or more of the framework regions. A mutation may be made in a framework region or constant domain to increase the half-life of the antibody. See, e.g., U.S. application Ser. No. 09/375,924, filed Aug. 17, 1999, herein incorporated by reference. A mutation in a framework region or constant domain may also be made to alter the immunogenicity of the antibody, to provide a site for covalent or non-covalent binding to another molecule, or to alter such properties as complement fixation. Mutations may be made in each of the framework regions, the constant domain and the variable regions in a single mutated antibody. Alternatively, mutations may be made in only one of the framework regions, the variable regions or the constant domain in a single mutated antibody.

In one embodiment, there are no greater than ten amino acid changes in either the VH or VL regions of the mutated antibody compared to the antibody prior to mutation. In a more preferred embodiment, there is no more than five amino acid changes in either the VH or VL regions of the mutated antibody, more preferably no more than three amino acid changes. In another embodiment, there are no more than fifteen amino acid changes in the constant domains, more preferably, no more than ten amino acid changes, even more preferably, no more than five amino acid changes.

Fusion Antibodies and Immunoadhesins

In another embodiment, a fusion antibody or immunoadhesin may be made which comprises all or a portion of an antibody of the present invention linked to another polypeptide. In a preferred embodiment, only the variable regions of the antibody are linked to the polypeptide. In another preferred embodiment, the VH domain of an antibody of the present invention is linked to a first polypeptide, while the VL domain of an antibody of this invention is linked to a second polypeptide that associates with the first polypeptide in a manner in which the VH and VL domains can interact with one another to form an antibody binding site. In another preferred embodiment, the VH domain is separated from the VL domain by a linker such that the VH and VL domains can interact with one another (see below under Single Chain Antibodies). The VH-linker-VL antibody is then linked to the polypeptide of interest. The fusion antibody is useful to directing a polypeptide to a gp120 expressing cell or tissue. The polypeptide may be a therapeutic agent, such as a toxin, growth factor or other regulatory protein, or may be a diagnostic agent, such as an enzyme that may be easily visualized, such as horseradish peroxidase. In addition, fusion antibodies can be created in which two (or more) single-chain antibodies are linked to one another. This is useful if one wants to create a divalent or polyvalent antibody on a single polypeptide chain, or if one wants to create a bispecific antibody.

The mutated antibodies may be screened for certain properties, such as improved binding of an antigen, such as a gp120 antigen.

Single Chain Antibodies

To create a single chain antibody, (scFv) the VH- and VL-encoding DNA fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see, e.g., Bird et al. (1988) Science 242:423-426; Huston et al. (1988) Proc. Natl. Acad. Sci. USA 85:5879-5883; McCafferty et al., Nature (1990) 348:552-554). The single chain antibody may be monovalent, if only a single VH and VL are used, bivalent, if two VH and VL are used, or polyvalent, if more than two VH and VL are used.

Kappabodies, Minibodies, Diabodies and Janusins

In another embodiment, other modified antibodies may be prepared using anti-HIV-1 gp120 encoding nucleic acid molecules. For instance, “Kappa bodies” (Ill et al., Protein Eng 10: 949-57 (1997)), “Minibodies” (Martin et al., EMBO J 13: 5303-9 (1994)), “Diabodies” (Holliger et al., Proc. Nat. Acad. Sci. USA 90: 6444-6448 (1993)), or “Janusins” (Traunecker et al., EMBO J 10: 3655-3659 (1991) and Traunecker et al. “Janusin: new molecular design for bispecific reagents” Int J Cancer Suppl 7:51-52 (1992)) may be prepared using standard molecular biological techniques following the teachings of the specification.

Chimeric Antibodies

In another aspect, bispecific antibodies can be generated. In one embodiment, a chimeric antibody can be generated that binds specifically to HIV-1 gp120 through one binding domain and to a second molecule through a second binding domain. The chimeric antibody can be produced through recombinant molecular biological techniques, or may be, physically conjugated together. In addition, a single chain antibody containing more than one VH and VL may be generated that binds specifically to HIV-1 gp120 and to another molecule. Such bispecific antibodies can be generated using techniques that are well known for example, in connection with (i) and (ii) see., e.g., Fanger et al. Immunol Methods 4: 72-81 (1994) and Wright and Harris, supra. and in connection with (iii) see, e.g., Traunecker et al. Int. J. Cancer (Suppl.) 7: 51-52 (1992).

Derivatized and Labeled Antibodies

An antibody or antibody portion of the invention can be derivatized or linked to another molecule (e.g., another peptide or protein). In general, the antibodies or portion thereof is derivatized such that the HIV-1 gp120 binding is not affected adversely by the derivatization or labeling. Accordingly, the antibodies and antibody portions of the invention are intended to include both intact and modified forms of the human anti-HIV-1 gp120 antibodies described herein. For example, an antibody or antibody portion of the invention can be functionally linked (by chemical coupling, genetic fusion, noncovalent association or otherwise) to one or more other molecular entities, such as another antibody (e.g., a bispecific antibody or a diabody), a detection agent, a cytotoxic agent, a pharmaceutical agent, and/or a protein or peptide that can mediate associate of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include these that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Another type of derivatized antibody is a labeled antibody. Useful detection agents with which an antibody or antibody portion of the invention may be derivatized include fluorescent compounds, including fluorescein, fluorescein isothiocyanate, rhodamine, 5-dimethylamine-1-napthalenesulfonyl chloride, phycoerythrin, lanthanide phosphors and the like. An antibody may also be labeled with enzymes that are useful for detection, such as horseradish peroxidase, β-galactosidase, luciferase, alkaline phosphatase, glucose oxidase and the like. When an antibody is labeled with a detectable enzyme, it is detected by adding additional reagents that the enzyme uses to produce a reaction product that can be discerned. For example, when the agent horseradish peroxidase is present, the addition of hydrogen peroxide and diaminobenzidine leads to a colored reaction product, which is detectable. An antibody may also be labeled with biotin, and detected through indirect measurement of avidin or streptavidin binding. An antibody may also be labeled with a predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

An antibody of the present invention may also be labeled with a radiolabeled amino acid. The radiolabel may be used for both diagnostic and therapeutic purposes. Examples of labels for polypeptides include, but are not limited to, the following radioisotopes or radionuclides—³H, ¹⁴C, ¹⁵N, ³⁵S, ⁹⁰Y, ⁹⁹Tc, ¹¹¹In, ¹²⁵I, ¹³¹I.

An antibody of the present invention may also be derivatized with a chemical group such as polyethylene glycol (PEG), a methyl or ethyl group, or a carbohydrate group. These groups may be useful to improve the biological characteristics of the antibody, e.g., to increase serum half-life or to increase tissue binding.

Characterization of Anti-HIV-1-gp120 Antibodies Class and Subclass of Antibodies

The class and subclass of antibodies of the present invention may be determined by any method known in the art. In general, the class and subclass of an antibody may be determined using antibodies that are specific for a particular class and subclass of antibody. Such antibodies are available commercially. The class and subclass can be determined by ELISA, Western Blot as well as other techniques. Alternatively, the class and subclass may be determined by sequencing all or a portion of the constant domains of the heavy and/or light chains of the antibodies, comparing their amino acid sequences to the known amino acid sequences of various class and subclasses of immunoglobulins, and determining the class and subclass of the antibodies.

In one embodiment of the invention, the antibody is a polyclonal antibody. In another embodiment, the antibody is a monoclonal antibody. The antibody may be an IgG, an IgM, an IgE, an IgA or an IgD molecule. In a preferred embodiment, the antibody is an IgG and is an IgG1, IgG2, IgG3 or IgG4 subtype. In a more preferred embodiment, the antibodies are subclass IgG2.

Pharmaceutical Compositions and Kits and Therapeutic Methods of Use

The invention also relates to a pharmaceutical composition for the treatment of a subject with an HIV-1 infection or for prophylactic administration (i.e., prevention) to a healthy subject, said composition comprises a therapeutically effective amount of an antibody of the invention.

Pharmaceutical compositions of this invention comprise any of the antibodies of the present invention, with any pharmaceutically acceptable carrier, adjuvant or vehicle. Pharmaceutically acceptable carriers, adjuvants and vehicles that may be used in the pharmaceutical compositions of this invention include, but are not limited to, any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The compositions of this invention may be in a variety of forms. These include, for example, liquid, semi-solid and solid dosage forms, such as liquid solutions (e.g., injectable and infusible solutions) dispersions or suspensions, tablets, pills, powders, liposomes and suppositories. The preferred form depends on the intended mode of administration and therapeutic application.

Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection. In another preferred embodiment, the composition is administered orally.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody of the present invention in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants, Prolonged absorption of injectable compositions can be brought about by including in the composition an agent that delays absorption, for example, monostearate salts and gelatin.

The antibodies of the present invention, as well as any other anti-viral agent, immunomodulator or immunostimulator, can be administered by a variety of methods known in the art, although for many therapeutic applications, the preferred route/mode of administration is subcutaneous, intramuscular, intravenous, intraperitoneal, or infusion. As will be appreciated by the skilled artisan, the route and/or mode of administration will vary depending upon the desired results.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, the antibody of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An exemplary, non-limiting range for a therapeutically or prophylactically effective amount of an antibody or antibody portion of the invention is 0.1-100 mg/kg, more preferably 0.5-50 mg/kg, more preferably 1-20 mg/kg, and even more preferably 1-10 mg/kg. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising the antibodies and the pharmaceutical compositions comprising these antibodies. A kit may include, in addition to the antibody or pharmaceutical composition, diagnostic or therapeutic agents. A kit may also include instructions for use in a therapeutic method. In another preferred embodiment, the kit includes the antibody or a pharmaceutical composition thereof and one or more anti-viral agents, immunomodulators and/or immunostimulators.

The antibodies of this invention may be administered to a healthy or HIV-infected subject either as a single agent or in combination with other anti-viral agents which interfere with the life cycle of HIV. By administering the compounds of this invention with other anti-viral agents, the therapeutic effect of these Mabs may be potentiated. For instance, the co-administered anti-viral agent can be one which targets early events in the life cycle of the virus, such as cell entry, reverse transcription and viral DNA integration into cellular DNA. Anti-HIV agents targeting such early life cycle events include, didanosite (ddI), dideoxycytidine (ddC), d4T, zidovudine (AZT), 3TC, 935U83, 1592U89, 524W91, polysulfated polysaccharides, sT4 (soluble CD4), ganiclovir, trisodium phosphonoformate, eflornithine, ribavirin, acyclovir, alpha interferon and tri-menotrexate. Additionally, non-nucleoside inhibitors of reverse transcriptase, such as TIBO, delavirdine (U90) or nevirapine, may be used to potentiate the effect of the antibodies of this invention, as may viral uncoating inhibitors, inhibitors of trans-activating proteins such as tat or rev, or inhibitors of the viral integrase. Furthermore, inhibitors of HIV protease may be co-administered.

Combination therapies according to this invention could exert an additive or synergistic effect in inhibiting HIV replication because each component agent of the combination acts on a different site of HIV replication. The use of such combination therapies may also advantageously reduce the dosage of a given conventional anti-retroviral agent which would be required for a desired therapeutic or prophylactic effect, as compared to when that agent is administered as a monotherapy. Such combinations may reduce or eliminate the side effects of conventional single anti-retroviral agent therapies, while not interfering with the anti-retroviral activity of those agents. These combinations reduce potential of resistance to single agent therapies, while minimizing any associated toxicity. These combinations may also increase the efficacy of the conventional agent without increasing the associated toxicity. Preferred combination therapies include the administration of a compound of this invention with AZT, ddI, ddC, d4T, 3TC, 935U83, 1592U89, 524W91, a protease inhibitor, existing antibodies against HIV-1 or a combination thereof.

Administering the antibodies of this invention as single agents or in combination with retroviral reverse transcriptase inhibitors, such as nucleoside derivatives, or other HIV aspartyl protease inhibitors, including multiple combinations comprising from 3-5 agents is preferred. The co-administration of the antibodies of this invention with retroviral reverse transcriptase inhibitors or HIV aspartyl protease inhibitors may exert a substantial additive or synergistic effect, thereby preventing, substantially reducing, or completely eliminating viral replication or infection or both, and symptoms associated therewith.

The antibodies of this invention can also be administered in combination with immunomodulators and immunostimulators (e.g., bropirimine, anti-human alpha interferon antibody, IL-2, GM-CSF, interferon alpha, diethyldithiocarbamate, tumor necrosis factor, naltrexone, tuscarasol, and rEPO); and antibiotics (e.g., pentamidine isethiorate) to prevent or combat infection and disease associated with HIV infections, such as AIDS, ARC and HIV-associated cancers.

When the antibodies of this invention are administered in combination therapies with other agents, they may be administered sequentially or concurrently to the subject. Alternatively, pharmaceutical compositions according to this invention may comprise a combination of an antibody of this invention and one or more therapeutic or prophylactic agents.

In one embodiment, the invention provides a method for treating a subject with an HIV-1 infection by administering an antibody of the present invention or an antigen-binding portion thereof to a patient in need thereof. In another embodiment, the invention provides a method for prophylactically treating a healthy subject by administering an antibody of the present invention or an antigen-binding portion thereof to said subject. In another embodiment, the invention provides a method of inhibiting the binding of HIV-1 virus to a T cell or a macrophage in a subject with an HIV-1 infection or who could get an HIV-1 infection comprising administering an effective amount to said subject of the antibody of this invention, or antigen-binding portion thereof. Any of the types of antibodies described herein may be used therapeutically or prophylactically (i.e. prevention). In a preferred embodiment, the subject is a human subject. The antibody may be administered to a non-human mammal with which the antibody cross-reacts (i.e. a primate, cynomologous or rhesus monkey) as an animal model of human disease. Such animal models may be useful for evaluating the therapeutic efficacy of antibodies of this invention.

The antibodies of this invention may also be used diagnostically to detect the presence of HIV-1 virus in a subject by detecting the presence of HIV-1 proteins (such as gp120) in the subject by ELISA, Western blot or any other known techniques for protein detection using an antibody, or an antigen-binding portion thereof. The presence of HIV-1 proteins in a subject could be done by detecting the presence of HIV-1 proteins in the subject's, for example, blood, serum, urine, tears, any other body fluid or secretion, tissue, organ, cells, etc.

In another embodiment, the antibody of the present invention is labeled with a radiolabel, an immunotoxin or a toxin, or is a fusion protein comprising a toxic peptide. The antibody or antibody fusion protein directs the radiolabel, immunotoxin, toxin or toxic peptide to the HIV-1 expressing cell. In a preferred embodiment, the radiolabel, immunotoxin, toxin or toxic peptide is internalized after the antibody binds to its binding partner on the surface of the cell.

In another embodiment, the antibody of the present invention is an antibody, or an antigen-binding portion thereof, that competes for binding with any one of the antibodies deposited as hybridomas expressing said antibodies with the ATCC, as detailed below in the “Biological Deposits” section, to an antigen (e.g., a gp120 antigen), such as the deposited antibody's antigen.

In another embodiment, the antibody of the present invention is an antibody, or an antigen-binding portion thereof, that comprises the heavy chain of any one of the antibodies produced by the deposited hybridomas, as detailed below in the “Biological Deposits” section.

In another embodiment, the antibody of the present invention is an antibody, or an antigen-binding portion thereof, that comprises the CDR1, CDR2 and CDR3 of the heavy chain of any one of the antibodies produced by a deposited hybridoma, as detailed below in the “Biological Deposits” section.

In another embodiment, the antibody of the present invention is an antibody, or an antigen-binding portion thereof, that comprises the heavy chain and the light chain of any one of the antibodies produced by a deposited hybridoma, as detailed below in the “Biological Deposits” section.

Method for Identifying a Region on HIV-1 gp120 for use as an HIV-1 Vaccine

In another aspect of this invention, it is provided a method of identifying a region on HIV-1 gp120 for use as an HIV-1 vaccine, said method comprising the steps or:
a) producing in a non-human mammal and isolating a human monoclonal antibody that binds gp120 and that has neutralizing activity for HIV-1; and
b) identifying an epitope (preferably linear epitope) on a V1 domain, a V2 domain and/or a V3 domain (or on a V1/V2/V3 domain and vicinity) of said gp120 that is bound by said antibody.

HIV-1 vaccine could, utilize, for example, full-length gp120 protein comprising a neutralizing epitope, portion thereof, a fusion protein comprising full-length gp120 protein, or portion thereof comprising a neutralizing epitope, or a peptide. The portion of the gp120 protein could be used as a vaccine by itself or part of a protein or another molecule. A pharmaceutical composition comprising said portion is provided herein as well.

Gene Therapy

The nucleic acid molecules of the antibodies of the instant invention may be administered to a patient in need thereof via gene therapy. The therapy may be either in vivo or ex vivo. In a preferred embodiment, nucleic acid molecules encoding both a heavy chain and a light chain are administered to a patient. In a more preferred embodiment, the nucleic acid molecules are administered such that they are stably integrated into the chromosome of B cells because these cells are specialized for producing antibodies. In a preferred embodiment, precursor B cells are transfected or infected ex vivo and re-transplanted into a patient in need thereof. In another embodiment, precursor B cells or other cells are infected in vivo using a virus known to infect the cell type of interest. Typical vectors used for gene therapy include liposomes, plasmids, or viral vectors, such as retroviruses, adenoviruses and adeno-associated viruses. After infection either in vivo or ex vivo, levels of antibody expression may be monitored by taking a sample from the treated patient and using any immunoassay known in the art and discussed herein.

In a preferred embodiment, the gene therapy method comprises the steps of administering an effective amount of an isolated nucleic acid molecule encoding the heavy chain encoding the heavy chain or the antigen-binding portion thereof of the human antibody or portion thereof and expressing the nucleic acid molecule. In another embodiment, the gene therapy method comprises the steps of administering an effective amount of an isolated nucleic acid molecule encoding the light chain or the antigen-binding portion thereof of the human antibody or portion thereof and expressing the nucleic acid molecule. In a more preferred method, the gene therapy method comprises the steps of administering an effective amount of an isolated nucleic acid molecule encoding the heavy chain or the antigen-binding portion thereof of the human antibody or portion thereof and an effective amount of an isolated nucleic acid molecule encoding the light chain or the antigen-binding portion thereof of the human antibody or portion thereof and expressing the nucleic acid molecules. The gene therapy method may also comprise the step of administering another anti-viral agent, immunomodulator and/or immunostimulator, as described above.

In order that this invention may be better understood, the following examples are set forth. These examples are for purposes of illustration only and are not to be construed as limiting the scope of the invention in any manner.

### EXAMPLE 1

### Human Monoclonal Antibodies that Specifically Bind HIV-1 GP120

### Materials and Methods

Recombinant Proteins and Synthetic Peptides

Soluble, rgp120s from the R5-tropic clade B primary isolates HIV_{SF162} (Cheng et al. (1989) Proc. Natl. Acad. Sci. U S A. 86:8575-8579) and HIV_{JR-FL} (Koyanagi, Y. et al. (1987) Science 236:819-822) were secreted from HEK293 (Graham et al. (1977) J. Gen. Virol. 36:59-72) cell lines stably expressing the recombinant proteins from pcDNA3.1zeo (Invitrogen). Coding sequences for these gp120s with were prepared by PCR from the molecular clones and fully sequenced. The sequence for rgp120_{JR-FL} was optimized at its initiation codon (Kozak (1989) J. Cell Biol. 108:229-241) and had a His6 affinity tag embedded in a run of Ala and Gly residues at its C-terminus.

In one case, a plasmid encoding a soluble HIV_{SF162} gp120 protein (SF162 is a CCR5-tropic isolate of HIV) was prepared in the following manner. The gp120 sequence of the primary HIV-1 isolate SF162 was amplified from the viral genomic DNA by PCR using primers 5′-agacatctagaatgagagtgaaggggatcagg-3′ (SEQ ID NO: 14) and 5′-gctccgaattcttattatcttttttctctctg-3′ (SEQ ID NO: 15). These primers introduced an XbaI site and an EcoRI site at sites flanking the gp120 gene. These sites were used to clone the PCR product into the pcDNA3.1 vector from Invitrogen (Invitrogen, Inc., San Diego, Calif.). A stable cell line was established by transfecting human 293 cell with this plasmid and selecting cells resistant to Zeocin. Cell clones secreting high concentrations of soluble rgp120 were identified by ELISAs on supernatant media, and grown in large scale.

Soluble rgp120s were purified to greater than 95% purity from cell culture media by lectin chromatography using Galanthus nivalis snowdrop agglutinin (Sigma Chem. Co.) as previously described (Gilljam et al. (1993) AIDS Res Hum Retroviruses May;9(5): 9:431-438), and were highly native as determined by reactivity with sCD4 and MAbs against conformational epitopes in V2 and the CD4 binding site.

Other soluble rgp120s were obtained from the NIH AIDS Research and Reference Reagent Program. These include gp120s derived from the X4-tropic clade B laboratory-adapted isolates HIV_{SF2} (#386), HIV_{IIIB} (#3926) and HIV_{MN} (#3927); the R5-tropic clade B primary isolate HIV_{BaL} (#4961); the R5-tropic clade E primary isolate HIV_{CM235} (#2968); and the clade E primary isolate HIV_{93TH975} (#3234).

Expression and purification of fusion proteins carrying HIV-1 variable domains attached to the C-terminus of an N-terminal fragment of a murine leukemia virus SU protein have been described, as well as the fusion proteins and methods of making them (Kayman, S. C. et al. (1994) J. Virol. 68:400-410 and Krachmarov et al. (2001) AIDS Research and Human Retroviruses Vol. 17, Number 18: 1737-1748, U.S. Pat. No. 5,643,756, issued Jul. 1, 1997, U.S. Pat. No. 5,952,474, issued Sep. 14, 1999). Wild type (JR-CSF circular in FIG. 6 and V3 fusion protein in FIGS. 2-3 and JR-CSF fusion protein) in FIG. 6B)) and linearized V3_{JR-CSF} fusion proteins (the linearized V3_{JR-CSF} fusion protein (JR-CSF linear in FIG. 6) is a mutant V3_{JR-CSF} fusion protein with the Cys at the N-terminal base of the V3 loop mutated to a Ser) and a fusion protein expressing the V1/V2_{SF162} domain (FIGS. 2 and 3) (U.S. Pat. No. 5,643,756, issued Jul. 1, 1997, U.S. Pat. No. 5,952,474, issued Sep. 14, 1999, Kayman, S. C. et al. (1994) J. Virol. 68:400-410 and Krachmarov et al. (2001) AIDS Research and Human Retroviruses Vol. 17, Number 18: 1737-1748) (see FIG. 3 for the region included) were used.

Synthetic peptides T15K (SEQ ID NO: 4), P130-1 (SEQ ID NO: 2), and P130-2 (SEQ ID NO: 3) were purchased from Bio-Synthesis, Inc. Lewisville, Tex. 75057. Peptides corresponding to various regions of the V3 loop from HIV_{MN} (full-length linear (“MN linear” (SEQ ID NO: 7)) (#1840); full-length circular (“MN cirucular” (SEQ ID NO: 8)) (#1841); MN 1-20 (SEQ ID NO: 9) (#1985); MN 11-30 (SEQ ID NO: 10) (#1986); MN 21-40 (SEQ ID NO: 11) (#1987); PND MN/IIIB MN 6-27+QR (SEQ ID NO: 12) (#864) and HIV_{IIIB} (SEQ ID NO: 13) (#1590) were obtained from the NIH AIDS Research and Reference Reagent Program.

Immunization and Hybridoma Isolation

Mice (XENOMOUSE® animals of the XMG2 strain, which are human gamma-2 κ antibody-producing transgenic mice), were immunized intradermally with SF162 rgp120 (recombinant gp120 (rgp120_{SF162})) (see, e.g., Mendez, M. et al. (1997) Nat. Genet. 15:146-156). Twenty μg of rgp120_{SF162} in the presence of Ribi adjuvant (MPL+TDM) was used to prime each XENOMOUSE® animal and fifteen μg of rgp120_{SF162} mixed with the same adjuvant was used to boost three times at 4-week intervals, with a final boost consisting of fifteen μg of rgp120_{SF162}, without adjuvant, given 4 days prior to fusion. In one experiment, immunizations were done with rgp120 that had been enzymatically deglycosylated by treatment with PNGase F (New England Biolabs). Specific antibodies to rgp120 were induced after several immunizations. XENOMOUSE® mice immunized with this antigen developed high titers of anti-gp120 antibodies after several immunizations. Splenocytes from immune XENOMOUSE® mice fused efficiently with Sp2/0 myelomas, allowing the isolation of large numbers of gp120-specific hybridomas.

Splenocytes from immunized XENOMOUSE® mice were harvested and fused with SP2/0 myeloma cells using standard techniques (see, e.g., Harlow and Lane Antibodies: A Laboratory Manual, Cold Spring Harbor Laboratory Press, Cold Spring Harbor, N.Y. (1990)). Briefly, splenocytes from XENOMOUSE® animals were harvested and fused with SP2/0 myeloma cells at a ratio of 5 spleen cells to 1 myeloma cell. Fusion was initiated by adding 1 ml of PEG/DMSO (Sigma P7306) to the cell mixture over 1 minute and stirring gently with the pipette for an additional minute. The cells were then diluted slowly by adding 10 mls of incomplete DMEM over a period of at least 10 minutes. The cells were then centrifuged at 400 g for 5 minutes, resuspended in HAT media and plated out in 96-well flat-bottom culture plates at concentration of 200,000 cells in 200 μl per well.

The plates were left undisturbed for seven days following the fusion. On day seven, the wells were fed by removing half the supernatant and 100 μl of HAT media were added to each well. Hybridomas were screened on day 12-14 by standard ELISA against rgp120_{SF162}.

Cells from positive wells were expanded and retested. Cultures that remained positive were subcloned until stable. Clonal hybridoma cell lines expressing human Mabs reactive with rgp120_{SF162} (recombinant gp120_{SF162}) were obtained. Cloning and sub-cloning were performed as follows. After screening, positive hybridomas were transferred to 48 well plates and expanded in HT media. Supernatants from the 48 wells were tested by ELISA against rgp120 and 2% BLOTTO alone. The repeatedly positive hybridomas were cloned and subcloned if desired, and rescreened by ELISA. Positive hybridomas were expanded to bulk culture for Ab purification and characterization. Antibodies were purified using protein A columns (Pharmacia, Inc. NJ), according to the manufacturer's specification.

Screening Assays

Hybridoma supernatants were screened by ELISAs as previously described (Pinter et al. (1993) AIDS Res. Hum. Retroviruses 9:985-996), using alkaline phosphatase-conjugated goat anti-human IgGs as the secondary antibody. In a typical experiment, 100 ng rgp120_{SF162} in 50 μl per well were coated onto 96-well ELISA plates in coating buffer (carbonate buffer, pH 9.8) at 4° C. overnight, and the wells were blocked with 100 μl 2% BLOTTO (Carnation powdered non-fat milk) for 1 h at 37° C. or overnight at 4° C. The plates were washed 3 times with PBS containing 0.05% Tween-20 (PBST), and 50 μl supernatant from the hybridomas culture were added into wells. After incubating for 2 h at 37° C., the plates were washed and second antibody (alkaline phosphatase conjugated goat anti-human antibody) added and incubated for 1 h at 37° C. After 3 washes with PBST, 50 μμl/well of AP developing reagent is added, and plates were read at OD405.

For binding inhibition studies, soluble CD4 (“sCD4”) and Mabs at 1 mg/ml were biotinylated for 4 hrs at room temperature with 1/8 volume of biotinamidocaproate N-hydroxysuccinimide ester (1 mg/ml in DMSO) (Sigma Chem Co.) followed by dialysis against PBS. Biotinylated probes and unlabelled competing reagents were mixed before adding to antigen-coated ELISA plates that were then processed normally using streptavidin-AP (Xymed) as the secondary reagent. Each biotinylated reagent was used at a concentration within its linear response range.

Measurement of HIV-Neutralization Activity

Neutralization activity of the human Mabs was measured in several different ways. The most useful assay was a single cycle infectivity assay, using the NL4-3 luciferase virus, pseudotyped with HIV-1 env. The NL4-3 luc virus has a defective env gene, and has the luc gene in place of nef. See Chen, B. K. et al. (1994) J. Virol. 68:-654-660. When complemented in trans with a functional env gene, the resulting virions transduce luc activity upon entry into susceptible cells. This assay is quite rapid, quantitative, and sensitive. Luciferase activity can be measured quickly and accurately as early as two days after infection, using a 96-well plate fluorometer, and the assay has a very large dynamic range.

HIV-1 Neutralization activity was determined with a single cycle infectivity assay using HIV-1 virions carrying Env-defective, luciferase-expressing HIV_{NL4-3} genomes (Chen et al. (1994) J. Virol. 68:654-660) that were pseudotyped with HIV_{SF162} Env as previously described (Krachmarov et al. (2001) AIDS Research and Human Retroviruses Vol. 17, Number 18: 1737-1748). Infections were carried out in 96 well format, and luciferase activity was determined 48-72 hrs post-infection using assay reagents from Promega and a microtiter plate luminometer (Dynex, Inc.). Routinely, 10,000 U-87-T4-CCR5 cells were plated out per well in a 96 well culture plate. One day later, d NL4-3 virus pseudotype was added at a concentration of 0.5 ng of p24 per ml, in the presence of 10 μg/ml polybrene. The cells were refed after 24 hrs with fresh medium plus polybrene, and allowed to grow for an additional 24-72 hours. Cells were then lysed with buffer provided in the Promega luciferase assay kit and luciferase activity measured by addition of luciferase substrate (Promega, Inc., Madison Wis.). Relative light units were then measured using a microtiter plate luminometer (Dynex, Inc., VA). Routinely, this results in 50,000-100,000 RLUs for control virus samples.

### Results

Efficient Generation of a Gp120-Specific Humoral Response in XENOMOUSE® Mice

Immunizing the XENOMOUSE® mice (G2 strain (“XMG2”)) with native recombinant gp120 derived from HIV_{SF162} resulted in robust antibody responses against multiple epitopes and domains of gp120, and allowed the efficient isolation of hybridomas producing gp120-specific human Mabs. The resulting Mabs were directed against multiple gp120 regions, and a number of these Mabs possessed strong neutralizing activities against the autologous SF162 strain. A broad range of epitopes were recognized by the isolated Mabs, including conserved conformational gp120 epitopes and both type-specific and cross-reactive epitopes. These results demonstrate the utility of the XENOMOUSE® system for identifying new and interesting epitopes of HIV-1, and suggest that this system may provide human Mabs suitable for immunotherapeutic applications, in detection of HIV-1 infection, prevention of HIV-1 invention and treatment of HIV-1 infection.

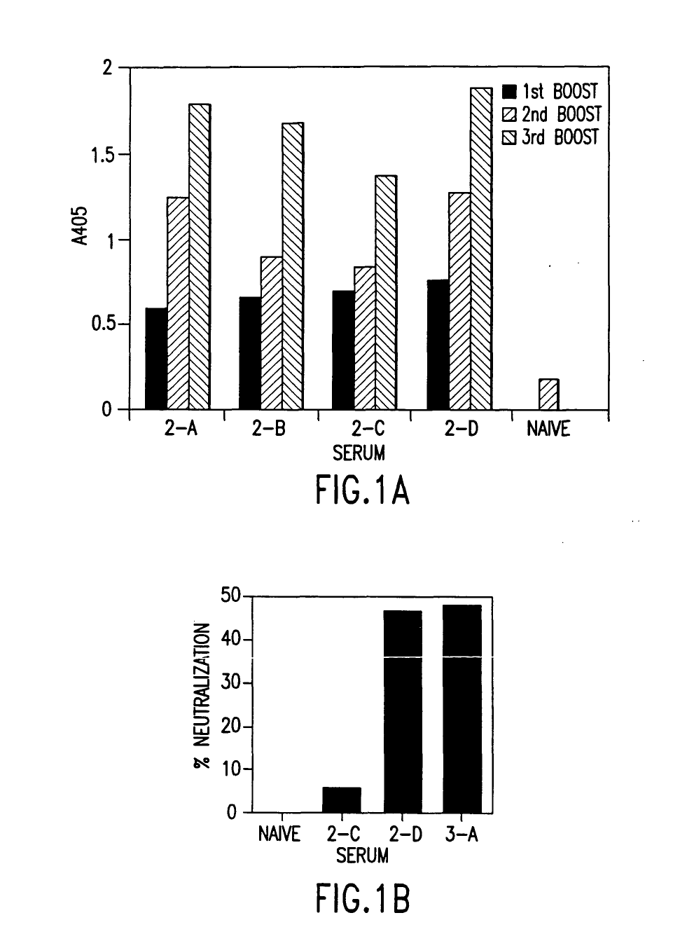

As shown in FIG. 1A, XENOMOUSE® mice, immunized with rgp120, produced rapid humoral responses against soluble HIV-1 gp120. FIG. 1A presents a typical profile of the humoral response of four XENOMOUSE® G2 animals immunized with soluble recombinant SF162 gp120 in the presence of Ribi adjuvant (MPL+TDM) All four XENOMOUSE® animals produced detectable gp120-specific antibodies after the first boost, and their antibody titers increased with subsequent immunizations. Sera of XENOMOUSE® mice immunized with this protocol often contained neutralizing activity against the autologous SF162 virus. Serum titers were determined by standard ELISA, using rgp120_{SF162} (50 ng/well) as target antigen. FIG. 1B shows results of a SF162 neutralization assay performed with a preimmune serum and three post-immunization sera of XENOMOUSE® mice (2-C, 2-D, 3-A) immunized with this protocol. The preimmune serum possessed no neutralizing activity, while two of three sera of XENOMOUSE® mice (2-D, 3-A) following immunizations neutralized SF162 with ND50s of approximately 1:25 dilution (FIG. 1B). These and other immunized animals were sacrificed and their splenocytes were fused with myeloma cells as described above.

The epitope specificities of the Mabs were analyzed by ELISAs using multiple antigens, including V1/V2 and V3 fusion proteins, synthetic peptides and rgp120s of multiple strains. These analyses showed that a large diversity of epitopes was recognized by these Mabs, including both type-specific and relatively conserved sequences. These epitopes included sites present in V1/V2 and V3 variable regions, as well as more conserved conformational structures.

Isolation and Initial Characterization of Gp120-Specific XENOMOUSE® Mabs

Splenocytes from immunized XENOMOUSE® mice fused efficiently with Sp2/0 myelomas, allowing the isolation of large numbers of gp120-specific hybridomas. These were initially screened by ELISA against the homologous rgp120 (rgp120_{SF162}) antigen, and positive wells were subcloned and rescreened for reactivity. Single cell clones obtained from positive subclones were then tested by ELISA for reactivity with fusion proteins expressing the gp120 variable domains, V1/V2 and V3 (Kayman et al. (1994) J. Virol. 68:400-410), and with rgp120_{SF162} reduced with DTT or not, in order to obtain preliminary mapping of the epitope specificities of the monoclonal antibodies produced. Representative data are presented in FIG. 2. Epitopes seen by the human Mabs from the XENOMOUSE® animals (“XENOMOUSE® Mabs”) included sites within and outside of the three variable domains tested. Eleven of these XENOMOUSE® Mabs were directed against the V1/V2 domain, and four were specific for the V3 domain. The XENOMOUSE® Mabs specific for these variable domains recognized linear epitopes, as indicated by their similar reactivities with native and reduced rgp120_{SF162} (FIG. 2, first and second panels). Of twenty XENOMOUSE® Mabs directed to gp120 sites outside the two major variable regions, seventeen did not react with reduced rgp120_{SF162}, indicating that they recognized disulfide-dependent conformational epitopes, while three had higher reactivity with rgp120_{SF162} after reduction. More precise definition of these epitopes is described below.

Characterization of XENOMOUSE® Mabs Directed Against Epitopes in V1/V2

The eleven XENOMOUSE® Mabs that reacted with the V1/V2 domain fusion protein (Kayman, S. C. et al. (1994) J. Virol. 68:400-410 and Krachmarov et al. (2001) AIDS Research and Human Retroviruses Vol. 17, Number 18: 1737-1748) (FIG. 2) retained reactivity with rgp120_{SF162} after reduction with DTT, suggesting that they might react with synthetic peptides. A 17-mer peptide matching the N-terminal region of the V2 domain (corresponding to the CaseA2 isolate (Wang et al. (1995) J. Virol. 69:2708-2715), which differs from the SF162 immunogen at two positions) was available (T15K (SEQ ID NO: 4)), and two overlapping 15-mer peptides matching the SF162 V1 domain were synthesized (FIG. 3B) (P130.1 and P130.2 ((SEQ ID Nos: 2 and 3, respectively)).

Ten of the SF162 V1/V2-reactive XENOMOUSE® Mabs reacted with the C-terminal V1 peptide, P130-2 (SEQ ID NO: 3), while the eleventh reacted with the V2 peptide (T15K (LEO ID NO: 4)) (FIG. 3A). These ten are Mab 35D10/D2: ATCC Accession No. PTA-3001, Mab 40H2/C7: ATCC Accession No. PTA-3006, Mab 43C7/B9: ATCC Accession No. PTA-3007, Mab 43A3/E4: ATCC Accession No. PTA-3005, Mab 45D1/B7: ATCC Accession No. PTA-3002, Mab 46E3/E6: ATCC Accession No. PTA-3008, Mab 58E1/B3: ATCC Accession No. PTA-3003, Mab 64B9/A6: ATCC Accession No. PTA-3004, Mab 69D2/A1 and Mab 82D3/C3. These ten Mabs (FIG. 3A) (Mab 35D10/D2: ATCC Accession No. PTA-3001, Mab 40H2/C7: ATCC Accession No. PTA-3006, Mab 43C7/B9: ATCC Accession No. PTA-3007, Mab 43A3/E4: ATCC Accession No. PTA-3005, Mab 45D1/B7: ATCC Accession No. PTA-3002, Mab 46E3/E6: ATCC Accession No. PTA-3008, Mab 58E1/B3: ATCC Accession No. PTA-3003, Mab 64B9/A6: ATCC Accession No. PTA-3004, Mab 69D2/A1 and Mab 82D3/C3 did not bind to a fusion protein comprising the V1/V2 domain of CaseA2 (Pinter et al. (1998) Vaccine 16: 1803-1808; Kayman, S. C. et al. (1994) J. Virol. 68:400-410 and Krachmarov et al. (2001) AIDS Research and Human Retroviruses Vol. 17, Number 18: 1737-1748, U.S. Pat. No. 5,643,756, issued Jul. 1, 1997, U.S. Pat. No. 5,952,474, issued Sep. 14, 1999). The XENOMOUSE® Mabs reactive with the C-terminal Vi peptide (P130.2 ((SEQ ID NO: 3)) did not react with the N-terminal V1 peptide (P130.1 (SEQ ID NO: 2)), indicating that the sequence KEMDGEIK (SEQ ID NO: 16), comprising the final four V1 residues and initial four residues of the central region, contained residues critical to these epitopes (a “V1 domain” could include amino acid residues just N-terminal and/or just C-terminal to the V1 domain; An antibody of this invention could recognize an epitope that is dependent on a V1 domain sequence or residue (s)). Two of these XENOMOUSE® Mabs reacted only weakly with the peptide (FIG. 3A); these antibodies also bound more weakly to rgp120, suggesting that they possessed low affinities. The epitopes of these two Mabs were more definitively mapped to the V1 region by the demonstration that the reactivity of these antibodies with the V1/V2 fusion protein and rgp120 was efficiently blocked by the V1 peptide (P130-2) (data not shown).

The general region corresponding to the V2 peptide recognized by 8.22.2 (8.22.3 and 8.22.2 are derived from two subclones of the original hybridoma clone) has previously been shown to contain epitopes recognized by several neutralizing rat Mabs (McKeating et al. (1993) J. Virol. 67:4932-4944), and to be part of the epitope of a very potently neutralizing chimpanzee Mab, C108G (Warrier et al. (1994) J. Virology 68:4636-4642). The epitopes of those non-human Mabs were localized to the N-terminal half of the peptide, and were highly type-specific for the HXB-2/HXB-10 sequences (C18G also recognized the BaL sequence (Vijh-Warrier, S. (1996) J. Virol. 70:4466-4473). The insensitivity of 8.22.2 binding to variation at two positions in the N-terminal region of T15K (SEQ ID NO: 4) suggested that the 8.22.2 epitope was localized to the C-terminal portion of that V2 peptide. This is a relatively conserved region, consistent with the broad cross-reactivity of this antibody within clade B (see FIGS. 8-9). These reactivity patterns suggested that the epitope of 8.22.2 involves different V2 amino acids than do previously described linear epitopes in V2. Mab 8.22.2 did not or does not bind to gp120 of HIV-1_{IIIB} or related clones, such as HXB2, HXB2d, or D10. A “V2 domain” could include amino acid residues just N-terminal and/or just C-terminal to the V2 domain. An antibody of this invention could recognize an epitope that is dependent on a V2 domain sequence or residue(s).

FIG. 10 shows V2 region sequences of gp120s tested for reactivity with Mab 8.22.2. The four gp120s tested that reacted with Mab 8.22.2 are SF162, CaseA2B, JR-FL and BaL. The three gp120s tested that did not react with Mab 8.22.2 are HXB2d, MN-ST and SF2. A sequence present in the region mapped by peptide T15K (SEQ ID NO: 4) that is conserved in the reactive sequences (QKEYALFYK (SEQ ID NO: 26)) is underlined.

Competition assays were performed to obtain information about the proximity of the epitopes of these newly isolated XENOMOUSE® Mabs with previously described epitopes in V1 and V2. Two of the anti-V1 XENOMOUSE® Mabs, one with high affinity (35D10/D2) and one with low affinity (43A3/E4), a previously described human Mab, derived from patients, against a conformational epitope in V2 (697D) (Gorny, M. K et al. (1994) J. Virol. 68:8312-8320) and sCD4 were biotinylated, and the ability of various Mabs to block their binding to SF162 rgp120 was determined (FIG. 5). As expected, neither 4117C, a human Mab derived from patients (“HuMabP”) directed against an epitope in the V3 domain, nor 5145A, a HuMabP directed against an epitope that overlaps the CD4 binding site (Cd4bs), blocked binding by any of the V1 or V2 reactive Mabs. None of the V1 or V2 reactive Mabs were effective at blocking the binding of sCD4, while the control HuMabP 5145A was highly effective. Thus, these V1 and V2 epitopes do not appear to overlap the CD4bs. All of the XENOMOUSE® Mabs reactive with the V1 domain peptide competed with both of the biotinlytated V1-specific XENOMOUSE® Mabs, consistent with the peptide binding data indicating the involvement of the KEMDGEIK sequence (SEQ ID NO: 16) in each of their epitopes. Neither of the biotinylated V1-specific XENOMOUSE® Mabs was competed by 8.22.2, the XENOMOUSE® Mabs directed against a linear V2 epitope, nor by two Mabs previously mapped to conformational V2 epitopes, the mouse Mab SC258 (Moore et al. (1993) J. Virol. 67:6136-6151) and the human Mab 697D (Gorny, M. K. et al. (1994) J. Virol. 68:8312-8320). Binding of biotinylated 697D was efficiently blocked by 8.22.2, but not by any of the V1-specific XENOMOUSE® Mabs. Thus, in the 3-dimensional structure of gp120, the linear V2 epitope is located in close proximity to the conformational V2 epitopes, but not to the V1 epitopes, despite the relative proximity of the V1 and V2 peptides in the primary sequence.

Characterization of XENOMOUSE® Mabs Directed Against Epitopes in V3

Four of the XENOMOUSE® Mabs were mapped to the V3 domain based on their reactivity with the V3_{JR-CSF} fusion protein (Kayman, S. C. et al. (1994) J. Virol. 68:400-410 and Krachmarov et al. (2001) AIDS Research and Human Retroviruses Vol. 17, Number 18: 1737-1748) (FIG. 6). JR-CSF is closely related to SF162. The epitopes of these Mabs were further localized by ELISA against a series of peptides corresponding to regions of the V3 domain of JR-CSF, MN and IIIB gp120s, and these epitopes were compared to those of a panel of HuMabPs against the V3 loop that have been isolated from HIV-1-infected human patients. The XENOMOUSE® Mabs mapped into two discrete groups (A and B) that were distinct from three groups (C, D, and E) into which the standard HuMabPs mapped (FIG. 6). a “V3 domain” could include amino acid residues just N-terminal and/or just C-terminal to the V3 domain. An antibody of this invention could recognize an epitope that is dependent on a V3 domain sequence or residue(s).

The most striking distinction was that while all of the standard HuMabPs reacted with the MN 1-20 peptide (SEQ ID NO: 9), corresponding to the N-terminal region and the crown (residues 15-18 (GPGR (SEQ ID NO: 17))) of the V3 loop, none of the XENOMOUSE® Mabs recognized this peptide. The group A XENOMOUSE® Mabs reacted with MN peptide 11-30 (SEQ ID NO: 10), implicating residues 21-30 (YTTKNIIGTI (SEQ ID NO: 25)) in their epitopes. Their failure to react with MN peptides 1-20 (SEQ ID NO: 9) and 21-40 (SEQ ID NO: 11) suggested that their epitopes spanned residue 20, near the crown of the loop. The reactivity of group A XENOMOUSE® Mabs with the PNDMN/IIIB (SEQ ID NO: 12) peptide but not HIV-1IIIB peptide (SEQ ID NO: 13) implicated Y21 and/or I27 in their epitopes (underlined in FIG. 6; numbering from the initial C of the MN V3 loop). Failure of these XENOMOUSE® Mabs to react with rgp120_{SF2} (see below) was consistent with an important role for Y21, which is the only position at which V3_{SF2} differs from the consensus in FIG. 6. Reactivity of group A XENOMOUSE® Mabs with the PNDMN/IIIB peptide (SEQ ID NO: 12), which incorporated the QR insertion following position 14 from the V3IIIB sequence, also suggested that group A epitopes are not sensitive to sequence in the region N-terminal to the crown of the loop. This QR insert is characteristic of V3IIIB and appeared to account at least in part for the type specificity of group E, but not group C and D, HuMabPs.

The Group B XENOMOUSE® Mab, 8.27.3, was distinguished from the others by its reactivity only with full length peptides, suggesting that it recognized a discontinuous or conformational epitope. Its reactivity with both the linear MN peptide and the linear form of the V3JR-CSF fusion protein indicated that the conformation of the 8.27.3 epitope was not dependent on the disulfide bond at the base of the V3 loop.

Characterization of XENOMOUSE® Mabs Epitopes Outside the Variable Domains

Most of the XENOMOUSE® Mabs isolated did not react with either of the variable region probes. Binding competition assays were performed to map the epitopes recognized by these antibodies. The ability of each XENOMOUSE® Mabs to inhibit binding of biotinylated sCD4 or a biotinylated XENOMOUSE® Mabs to rgp120_{SF162} in ELISA was determined (FIG. 7). Six XENOMOUSE® Mabs (Conf.-gp120-A or Conf A, CD4bs or CD4bs) and a control HuMabP (5145A) efficiently blocked binding of sCD4 to gp120, indicating that they were directed against an epitope or epitopes overlapping the CD4bs of gp120. All of these XENOMOUSE® Mabs recognized a disulfide bond-dependent epitope (FIG. 2), consistent with the conformational nature of the CD4bs and standard epitopes that mediate inhibition of sCD4 binding (Thali, M., C. et al. (1992) J. Virol. 66:5635-5641).

Eleven XENOMOUSE® Mabs directed against disulfide bond-dependent epitopes did not inhibit binding of sCD4. All of these Mabs did block binding by one member of the group, 63G3/E2, but did not block binding by one of the XENOMOUSE® Mabs directed against the CD4bs, 38G3/A9 (FIG. 7). These XENOMOUSE® Mabs therefore constituted a distinct competition group (Conf-gp120-B or Conf B). Two of these XENOMOUSE® Mabs inhibited 63G3/E2 only partially, which might reflect either lower affinity or reactivity with an epitope that only partially overlapped the other Conf-gp120-A epitopes.

The three XENOMOUSE® Mabs that were reactive with reduced rgp120 but neither the V1/V2 nor the V3, fusion proteins constituted a third competition group (gp120-C). Each of these Mabs inhibited 97B1/E8 binding, but did not significantly block binding by sCD4 or XENOMOUSE® Mabs directed against CD4bs or Conf-gp120-B epitopes (FIG. 7). The XENOMOUSE® Mabs directed against gp120-C epitopes were all isolated from mice immunized with rgp120 that had been deglycosylated with PNGase F. The binding of these antibodies to gp120 was enhanced upon reduction of disulfide bonds (FIG. 1), suggesting that their epitopes are exposed by denaturation of the glycosylated molecules.

Extent of Conservation of Epitopes Recognized by XENOMOUSE® Mabs

The extent to which these XENOMOUSE® Mabs were cross-reactive was explored by performing ELISA against a panel of eight rgp120s (FIG. 8). Gp120s derived from three R5-tropic clade B isolates, three X4-tropic clade B viruses and two clade E isolates were used.

The V1-specific XENOMOUSE® Mabs were all highly specific for rgp120_{SF162}, consistent with this domain being the most highly variable in region in gp120 (Human Retroviruses and AIDS, 1996: A Compilation and Analysis of Nucleic Acid and Amino Acid Sequences, edited by. Myers, G., B. Korber, B. Foley, K. T. Jeang, J. W. Mellors, and S. Wain-Hobson (1996) Los Alamos National Laboratory, Los Alamos, N.Mex., published by Theoretical Biology and Biophysics Group T-10, Mail Stop K710, Los Alamos, N.Mex. 87545 (http://hiv-web.lanl.gov/)). The V2-specific XENOMOUSE® Mab, 8.22.2, reacted with all three R5-tropic (i.e, CCR5-tropic) clade B gp120s but with none of the X4-tropic (i.e, CXCR4-tropic) clade B gp120s, consistent with both the existence of regions of significant sequence similarity (Human Retroviruses and AIDS, 1996: A Compilation and Analysis of Nucleic Acid and Amino Acid Sequences, edited by Myers, G., B. Korber, B. Foley, K. T. Jeang, J. W. Mellors, and S. Wain-Hobson (1996) Los Alamos National Laboratory, Los Alamos, N.Mex., published by Theoretical Biology and Biophysics Group T-10, Mail Stop K710, Los Alamos, N.Mex. 87545 (http://hiv-web.lanl.gov/)) and the presence of determinants of tropism within this variable domain (Morikita T, M. Y. et al. (1997) AIDS Res Hum Retroviruses:1291-1299, Ogert R A et al. J. Virol.:5998-6006, Shieh J T et al. (2000) J. Virol.:693-701, Vella C, K. D. et al. (1999) AIDS Res Hum Retroviruses:1399-1402). The V3-specific XENOMOUSE® Mabs recognized from four to five gp120s within clade B with no obvious bias with respect to co-receptor usage; only the Group B XENOMOUSE® Mabs (such as 8.27.3) recognized rgp120_{SF2}.

The XENOMOUSE® Mabs directed against epitopes outside of these variable domains were highly cross-reactive. Four of the CD4bs-specific XENOMOUSE® Mabs recognized all six of the clade B rgp120s, one recognized five, and one (the only one derived from immunization with deglycosylated gp120) was type-specific for SF162. The Conf.-gp120-B XENOMOUSE® Mabs reacted with from three to seven rgp120s, in most cases including at least one of the clade E proteins. The gp120-C XENOMOUSE® Mabs were also cross-reactive, recognizing three to six clade B rgp120s. The variation in recognition patterns of antibodies within most of these groupings suggested that these Mabs identified multiple epitopes in each of these epitope clusters.

Neutralizing Activity of XENOMOUSE® Mabs

Each of the XENOMOUSE® Mabs were tested for the ability to neutralize SF162 HIV-1 virus. A single cycle infection assay was used that employs virions bearing SF162 envelope proteins and carrying a defective HIV-1 genome that expresses luciferase. Neutralization was seen for at least one of the XENOMOUSE® Mabs directed against each of four epitope clusters, the V1, V2 and V3 variable domains and the CD4bs (FIGS. 4 and 9). None of the XENOMOUSE® Mabs against the conformational gp120-B domain or the linear gp120-C domain possessed neutralizing activity, even at 200 μg/ml (FIG. 9). This lack of neutralization may reflect either a lack of exposure of these domains in intact virions, or the lack of a function for these regions that can be interfered with by antibody binding.

The anti-V1 XENOMOUSE® Mabs all possessed potent neutralizing activities for the SF162 strain, with ND50s ranging from below about 0.3 μg/ml to about 4.5 μg/ml (FIG. 9). Ten of the anti-V1/V2 Mabs (which are 35D10/D2, 40H2/C7, 43A3/E4, 43C7/B9, 45D1/B7, 46E3/E6, 58E1/B3 and 64B9/A6, 69D2/A1 and 82D3/C3) neutralized SF162, many with quite potent end points (FIG. 5). All ten of those antibodies were specific for linear V1 epitopes.

The V2-specific XENOMOUSE® Mabs, 8.22.2, had less potent neutralizing activity, with an ND50 of approximately 48 μg/ml. These activities were all more potent than that of the control anti-V2 HuMabP, 697D, which had an ND50 of about 80 μg/ml. The V3-specific XENOMOUSE® Mabs varied widely in their neutralizing, potencies. Mab 8.27.3 had the strongest neutralizing activity of all the XENOMOUSE® Mabs, with an ND50 of about 0.11 μg/ml, while 8E11/A8 had an ND50 of about 2.6 μg/ml. However, two additional V3-specific XENOMOUSE® Mabs with the same reactivity pattern as 8E11/A8, 6.1 and 6.7, had no detectable neutralizing activity at a concentration of 50 μg/ml. Four of the XENOMOUSE® Mabs directed against epitopes in the CD4 binding site also possessed moderate neutralizing activities, with ND50s in the range of 30-60 μg/ml. Two additional XENOMOUSE® Mabs against this domain did not neutralize at 200 μg/ml. The variability in neutralization potencies of the XENOMOUSE® Mabs directed against these neutralization domains may be due to different affinities or to subtle differences in the structure and functional roles of their epitopes.

The hypervariable V1 loop of gp120 was an immunodominant region for the panel of XENOMOUSE® Mabs isolated and described above, and all of antibodies directed against this domain had potent type-specific neutralizing activity. This is the first description of Mabs against the V1 domain (B. Korber, C. B., B. Haynes, R. Koup, C. Kuiken, J. Moore, B. Walker, D. Watkins (2000) HIV Molecular Immunology. Los Alamos National Laboratory, Los Alamos, N.Mex.; see also http//hiv-web.lanl.gov and http//hiv-web.lanl.gov/immunology). A previous study examining the humoral response of three laboratory workers infected with the laboratory adapted X4-tropic HIV_{IIIB} virus reported that the V1 region was the immunodominant target of neutralizing antibodies against the infecting strain (Pincus, S. H. et al. (1994) J. Clin. Invest. 93:2505-2513), consistent with the results of the current study. The relatively potent neutralizing activities of the V1-specific Mabs described above demonstrates that this region is also a potent neutralizing target in at least one R5-tropic virus, suggesting that such antibodies may be important components of the in vivo neutralizing humoral response.

Although only a single XENOMOUSE® Mab directed against the V2 domain, 8.22 (8.22.2 is a subclone of 8.22.3), was isolated in this study, this antibody was directed against a unique and interesting epitope. Unlike other Mabs against linear epitopes in V2 (McKeating, J. A. et al. (1993) J. Virol. 67:4932-4944, Shotton et al., J. Virol. 69: 222-230). 8.22.2 (a subclone of 8.22) was moderately cross-reactive, recognizing all three clade B R5-tropic rgp120s that were tested (FIG. 8). Also, 8.22.2 did not bind the gp120 of HIV-1_{1IIIB}, an X4 Clade B isolate (FIG. 8). Other cross-reactive Mabs directed against V2 have been reported, but are directed against conformational epitopes that depend on the disulfide-bonded structure of the domain (Fung, M. S. C. et al. (1992) J. Virol. 66:848-856, Gorny, M. K. et al. (1994) J. Virol. 68:8312-8320, Ho, D. D. et al. (1991) Proc. Natl. Acad. Sci. USA. 88:8949-8952). Furthermore, 8.22.2 had significant neutralizing activity against the R5-tropic HIV_{SF162} isolate, being over ten-fold more potent than 697D, the V2-directed Human Mab previously reported to neutralize such virus isolates (Gorny, M. K. et al. (1994) J. Virol. 68:8312-8320). This result was consistent with the high potency of the chimp Mab C108G, which mapped to a glycan-dependent epitope localized in the same region of V2.

The repertoire of V3 epitopes identified in this study was also interesting. First, the V3-reactive XENOMOUSE® Mabs were moderately cross-reactive, with the more potent of the two neutralizing XENOMOUSE® Mabs (group B, 8.27.3) recognizing five of the six clade B rgp120s tested, and the other neutralizing-V3-specific XENOMOUSE® Mabs (group A, 8E11/A8), recognizing, four of the six clade B rgp120s. The rgp120 not recognized by either group was from the HIV-1IIIB isolate, which has an immunologically distinct V3 domain. The other rgp120 not recognized by the group A XENOMOUSE® Mabs was from HIV_{SF2}. The potent group B XENOMOUSE® Mab (8.27.3) was also unique in that it reacted with only full length V3 loop peptides. These epitope differences may result in part from differences in the immune repertoire between the XENOMOUSE® mouse strain used and humans. However, HIV_{SF2} was found to be unusually resistant to V3-directed neutralizing antibodies affinity purified from human patient sera (Krachmarov et al. (2001) AIDS Research and Human Retroviruses Vol. 17, Number 18: 1737-1748). This suggests the possibility that the group A epitopes may actually be representative of neutralizing V3 targets seen in infected patients.

The majority of the XENOMOUSE® Mabs isolated in this study were directed against epitopes not contained within the V1, V2, or V3 variable domains. These antibodies were directed against conserved epitopes, which were conformational, except for three induced by immunization with deglycosylated rgp120_{SF162}. Binding competition studies separated the XENOMOUSE® Mabs directed against conformational epitopes into two groups, one of which corresponded to the previously described CD4bs cluster (Cordell, J. et al. (1991) Virology 185:72-79., Ho, D. D. et al. (1991) J. Virol. 65:489-493, McKeating, J. A. et al. (1992) Virology 190:134-142., Thali et al. (1992) J. Virol. 66:5635-5641, Tilley et al. (1991) Human monoclonal antibodies against the putative CD4 binding site and the V3 loop of HIV gp120 act in concert to neutralize virus. VII Intl. Conf. on AIDS. abstr. 70: Florence, Italy). Neither of these groups overlapped with the XENOMOUSE® Mabs against reduction-insensitive epitopes, which were preferentially presented by denatured rgp120. Some of the XENOMOUSE® Mabs against CD4bs epitopes had moderate neutralization activity, while none of the XENOMOUSE® Mabs against the other cluster of conformational epitopes had any neutralization activity. One face of soluble monomeric gp120 is occluded in the native trimeric Env complex (Kwong et al. (1998) Nature 393:648-659, Rizzuto, C. D. et al. (1998) Science :1949-1953, Wyatt, R. et al. (1998) Nature 393:705-711), and it is possible that the latter class of XENOMOUSE® Mabs were directed against epitopes on this surface.

Use of HIV-1 immunogens other than rgp120_{SF162} and/or other screening methods may allow the isolation of more effective neutralizing XENOMOUSE® Mabs against already identified domains as well as neutralizing Mabs against completely new targets. Different rgp120 immunogens may induce responses against different classes of conserved and variable region epitopes. It may be possible to avoid the isolation of Mabs against the occluded face of gp120 by immunizing and/or screening with oligomeric Env complexes, such as recently described stabilized trimeric forms of HIV-1 Env proteins (Binley et al. (2000) J. Virol. 74:627-643, Yang, X. et al. (2000) J. Virol. 74:5716-5725), or native Env complexes expressed on viral particles or cell surfaces. A direct screen for neutralization activity that has been developed may be particularly useful for focussing on the most relevant Mabs. Antigens consisting of trimeric Env complexes, either soluble or membrane-associated, may be effective immunogens for neutralization targets that are poorly expressed, if at all, on the gp120 monomer.

As demonstrated herein, the XENOMOUSE® system provides a useful approach for isolating human monoclonal antibodies against HIV-1 Env. The availability of transgenic mice that produce fully human antibodies, together with the development of novel immunogens and functional screening assays, should facilitate the more complete mapping of targets for the neutralization of HIV-1 infection, and should facilitate the isolation of Human Mabs with potential clinical utility as immunotherapeutic agents against HIV-1.

Biological Deposits

The following hybridomas (which are mouse hybridomas) expressing the antibodies as indicated below—
cell line 35D10/D2 (Mab 35D10/D2): ATCC Accession No. PTA-3001,
cell line 40H2/C7 (Mab 40H2/C7): ATCC Accession No. PTA-3006,
cell line 43C7/B9 (Mab 43C7/B9): ATCC Accession No. PTA-3007,
cell line 43A3/E4 (Mab 43A3/E4): ATCC Accession No. PTA-3005,
cell line 45D1/B7 (Mab 45D1/B7): ATCC Accession No. PTA-3002,
cell line 46E3/E6 (Mab 46E3/E6): ATCC Accession No. PTA-3008,
cell line 58E1/B3 (Mab 58E1/B3): ATCC Accession No. PTA-3003,
cell line 64B9/A6 (Mab 64B9/A6): ATCC Accession No. PTA-3004, and
cell line 8.27.3 (also known as cell line Abx 8.27.3) (Mab 8.27.3 (also known as Mab Abx 8.27.3)): ATCC Accession No. PTA-3009,
   were deposited with the American Type Culture Collection (“ATCC”), 10801 University Boulevard, Manassas, Va. 20110-2209, USA, on Feb. 2, 2001 (the ATCC confirmed receipt of these 9 hybridomas on Feb. 2, 2001 by email), and given the above-indicated ATCC Accession Numbers.

The following hybridoma (which is mouse hybridoma) expressing the antibody as indicated below
cell line 8.22.2 (Mab 8.22.2): ATCC Accession No.,
   was deposited with the American type Culture Collection (“ATCC”), 10801 University Boulevard, Manassas, Va. 20110-2209, USA, on Jan. 24, 2002, and given the above-indicated ATCC Accession Number.

The following hybridoma (which is a mouse hybridoma) expressing the antibody as indicated below
cell line 8E11/A8 (Mab 8E11/A8): ATCC Accession No.,
   was deposited with the American Type Culture Collection (“ATCC”), 10801 University Boulevard, Manassas, Va. 20110-2209, USA; on Jan. 25, 2002, and given the above-indicated ATCC Accession Number.

In one embodiment of this invention, the antibody of the present invention is an antibody that competes for binding of any one of the antibodies, described above in this section (Biological Deposits), deposited with the ATCC, to an antigen (could be a gp120 antigen), such as the deposited antibody's antigen.

In another embodiment, the antibody of the present invention is an antibody that comprises the heavy chain of any one of the antibodies, described above in this section (Biological Deposits), deposited with the ATCC.

In another embodiment, the antibody of the present invention is an antibody that comprises the CDR1, CDR2 and CDR3 of the heavy chain any one of the antibodies, described above in this section (Biological Deposits), deposited with the ATCC. The assignment of amino acids to each CDR domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

In another embodiment, the antibody of the present invention is an antibody that comprises the heavy chain and the light chain of any one of the antibodies, described above in this section (Biological Deposits), deposited with the ATCC.

All publications, patens and patent applications cited in this specification are herein incorporated by reference as if each individual publication, patent or patent application were specifically and individually indicated to be incorporated by reference.

Equivalents

The invention may be embodied in other specific forms without departing from the spirit or essential characteristics thereof. The foregoing embodiments are therefore to be considered in all respects illustrative of, rather than limiting on, the invention disclosed herein.

1. An isolated human antibody or antigen-binding portion thereof that specifically binds to HIV-1 gp120 protein and that has HIV-1 neutralizing activity, wherein said antibody or antigen-binding portion thereof recognizes an epitope on a V1/V2 domain of HIV-1 gp120, wherein said epitope is dependent on the presence of a sequence in the V1 loop.

2-9. (Canceled)

10. The isolated human antibody or antigen-binding portion thereof according to claim 1, wherein said antibody or antigen binding portion thereof has HIV-1_{SF162} neutralizing activity.

11. The isolated human antibody or antigen-binding portion thereof according to claim 1, wherein said antibody or antigen binding portion thereof recognizes a linear epitope on a V1 domain of HIV-1_{SF162} gp120.

12. The isolated human antibody or antigen-binding portion thereof according to claim 10, wherein said antibody or antigen binding portion thereof recognizes a linear epitope on a V1 domain of HIV-1_{SF162} gp120.

13. The isolated human antibody or antigen-binding portion thereof according to claim 1, wherein said antibody binds to a peptide consisting of SEQ ID NO: 3.

14. The isolated human antibody or antigen-binding portion thereof according to claim 13, wherein said antibody does not bind to a peptide consisting of SEQ ID NO: 2.

15. The isolated human antibody or antigen-binding portion thereof according to claim 10, wherein said HIV-1_{SF162} neutralizing activity is approximately as strong as the HIV-1_{SF162} neutralizing activity of human monoclonal antibody selected from the group consisting of 45D1/B7, secreted by a hybridoma designated by ATCC Accession Number PTA-3002, 58E1/B3, secreted by a hybridoma designated by ATCC Accession Number PTA-3003 and 64B9/A6, secreted by a hybridoma designated by ATCC Accession Number PTA-3004.

16. The isolated human antibody or antigen-binding portion thereof according to claim 1, wherein the human antibody is a human monoclonal antibody.

17-18. (Canceled)

19. A hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003) and cell line 64B9/A6 (ATCC Accession Number PTA-3004).

20. The human monoclonal antibody produced by a hybridoma cell line according to claim 19, or an antigen-binding portion thereof.

21. (Canceled)

22. The isolated human antibody or antigen-binding portion thereof according to claim 1, wherein said human antibody comprises a heavy chain CDR1, CDR2 and CDR3 from the antibody selected from the group consisting of:
a) a human monoclonal antibody produced by a hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003) and cell line 64B9/A6 (ATCC Accession Number PTA-3004);
b) a human antibody produced by the hybridoma cell line designated 8.22.2 and having ATCC Accession Number PTA-4007; and
c) a human antibody produced by a hybridoma cell line selected from the group consisting of: cell line 8.27.3 (ATCC Accession Number PTA-3009) and cell line 8E11/A8 (ATCC Accession Number PTA-4012).

23. The isolated human antibody or antigen-binding portion thereof according to claim 1, wherein said human antibody comprises a heavy chain of the human antibody according to claim 20.

24. A nucleic acid molecule comprising a nucleotide sequence that encodes the heavy chain of the antibody according to claim 20.

25. A nucleic acid molecule comprising a nucleotide sequence that encodes the light chain of the antibody according to claim 20.

26. (Canceled)

27. A host cell transformed with the nucleic acid according to claim 24.

28-30. (Canceled)

31. An isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120.

32. The isolated human antibody or antigen-binding portion thereof according to claim 31, wherein said antibody or antigen-binding portion thereof recognizes a linear epitope on a V2 domain of HIV-1_{SF162} gp120.

33. The isolated human antibody or antigen-binding portion thereof according to claim 31, wherein said antibody or antigen binding portion thereof has HIV-1_{SF162} neutralizing activity.

34. (Canceled)

35. The isolated human antibody or antigen-binding portion thereof according to claim 31, wherein the human antibody is a human monoclonal antibody.

36. The isolated human antibody or antigen-binding portion thereof according to claim 31, wherein said human antibody binds to at least three CCR5 Clade B HIV-1 gp120 proteins.

37. The isolated human antibody or antigen-binding portion thereof according to claim 31, wherein said human antibody binds to a peptide consisting of the sequence of SEQ ID NO: 4.

38. The isolated human antibody or antigen-binding portion thereof according to claim 31, wherein said human antibody, wherein said antibody does not bind to a gp120 of HIV-1 IIIB, HBX2, HBX2d or BH10.

39. A hybridoma cell line designated 8.22.2 and having ATCC Accession Number PTA-4007.

40. A human antibody produced by the hybridoma cell line according to claim 39, or antigen-binding portion thereof.

41. The isolated human antibody or antigen-binding portion thereof according to claim 31, wherein said antibody or antigen-binding portion thereof competes with the antibody according to claim 40 for binding to an antigen bound by the antibody according to claim 40.

42. (Canceled)

43. The isolated human monoclonal antibody or antigen-binding portion thereof according to claim 35, wherein said human monoclonal antibody comprises a heavy chain of the antibody according to claim 40.

44. The isolated human monoclonal antibody or antigen-binding portion thereof according to claim 35, wherein said human antibody comprises a heavy chain CDR1, CDR2 and CDR3 from the antibody according to claim 40.

45. A nucleic acid molecule comprising a nucleotide sequence that encodes the heavy chain of the antibody according to claim 40.

46. A nucleic acid molecule comprising a nucleotide sequence that encodes the light chain of the antibody according to claim 40.

47. (Canceled)

48. A host cell transformed with a nucleic acid according to claim 45.

49-51. (Canceled)

52. The isolated human antibody or antigen-binding portion thereof according any one of claims 1 or 31, wherein the antibody or portion thereof has HIV-1 neutralizing activity in vivo.

53. The isolated human antibody or antigen-binding portion thereof according to any one of claims 1 or 31, wherein said antibody has neutralizing activity for more than one primary isolate of HIV-1.

54. (Canceled)

55. The isolated human antibody or antigen-binding portion thereof according to any of claims 53, wherein said more than one primary isolate of HIV-1 are members of more than one clade.

56. An isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

57. The isolated human monoclonal antibody or antigen-binding portion thereof according to claim 56, wherein said V3 region is the V3 region of HIV-1_{SF162} gp120.

58. A hybridoma cell line selected from the group consisting of: cell line 8.27.3 (ATCC Accession Number PTA-3009) and cell line 8E11/A8 (ATCC Accession Number PTA-4012).

59. The human antibody produced by a hybridoma cell line according to claim 58, or antigen-binding portion thereof.

60. (Canceled)

61. The isolated human monoclonal antibody or antigen-binding portion thereof according to claim 56, wherein said human antibody comprises a heavy chain CDR1, CDR2 and CDR3 from the antibody according to claim 59.

62. The isolated human monoclonal antibody or antigen-binding portion thereof according to claim 56, wherein said antibody comprises a heavy chain of a human antibody according to claim 59.

63. The isolated human monoclonal antibody or antigen-binding portion thereof according to claim 56, wherein said antibody or antigen-binding portion thereof competes with a human antibody according to claim 59 for binding to an antigen bound by said antibody according to claim 59.

64. The isolated human monoclonal antibody or antigen-binding portion thereof according to claim 56, wherein said antibody has HIV-1 neutralizing activity.

65. The isolated human monoclonal antibody or antigen-binding portion thereof according to claim 64, wherein said antibody has HIV-1_{SF162} neutralizing activity.

66. The isolated human monoclonal antibody or antigen-binding portion thereof according to claim 64, wherein the antibody or portion thereof has HIV-1 neutralizing activity in vivo.

67. The isolated human monoclonal antibody or antigen-binding portion thereof according to claim 64, wherein said antibody has neutralizing activity for more than one primary isolate of HIV-1.

68. The isolated human antibody or antigen-binding portion thereof according to claim 67, wherein said for more than one primary isolate of HIV-1 are members of more than one clade.

69. The isolated human antibody or antigen-binding portion thereof according to any one of claims 1, 31 or 56, wherein said antibody or portion thereof inhibits the binding of HIV-1 gp120 to human CXCR4 receptor.

70. The isolated human antibody or antigen-binding portion thereof according to any one of claims 1, 31 or 56, wherein said antibody or portion thereof inhibits the binding of HIV-1 gp120 to human CCR5 receptor.

71. A nucleic acid molecule comprising a nucleotide sequence that encodes the heavy chain of the antibody according to claim 59.

72. A nucleic acid molecule comprising a nucleotide sequence that encodes the light chain of the antibody according to claim 59.

73. (Canceled)

74. A host cell transformed with a nucleic acid according to claim 71.

75-77. (Canceled)

78. The isolated human monoclonal antibody or antigen-binding portion thereof according any one of claims 16, 35 or 56, wherein the antibody or portion thereof is an immunoglobulin G (IgG), an IgM, an IgE, an IgA or an IgD molecule, or is derived therefrom.

79-84. (Canceled)

85. The isolated human monoclonal antibody or antigen-binding portion thereof according to claim 16, 35 or 56 wherein the antibody or portion thereof is labeled.

86-87. (Canceled)

88. The isolated human monoclonal antibody or antigen-binding portion thereof according to claim 85, wherein the label is selected from the group consisting of a radiolabel, an enzyme label, a toxin and a magnetic agent.

89. (Canceled)

90. The isolated antigen-binding portion thereof according to any one of claims 1, 31 or 56, wherein said antigen-binding fragment is an Fab fragment, an F(ab′)₂ fragment or an F_{V} fragment.

91. The isolated human monoclonal antibody or antigen-binding portion thereof according to claim 16, 35 or 56 wherein the antibody is a single chain antibody.

92-93. (Canceled)

94. The isolated human monoclonal antibody or antigen-binding portion thereof according to claim 16, 35 or 56 wherein the antibody is a chimeric antibody.

95-96. (Canceled)

97. The chimeric antibody according to claim 94, wherein the chimeric antibody comprises framework regions and CDR regions from different human monoclonal antibodies.

98-101. (Canceled)

102. The chimeric antibody according to claim 94, wherein the chimeric antibody is bispecific.

103. (Canceled)

104. The isolated human monoclonal antibody or antigen-binding portion thereof according to claim 16, 35 or 56 wherein the antibody or portion thereof is derivatized.

105-106. (Canceled)

107. The isolated human monoclonal antibody or antigen-binding portion thereof according to claim 104, wherein the antibody or portion thereof is derivatized with polyethylene glycol, at least one methyl or ethyl group or at least one carbohydrate moiety.

108. (Canceled)

109. A composition comprising an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9, and a pharmaceutically acceptable carrier.

110. The composition according to claim 109 further comprising one or more additional therapeutic agents.

111. The composition according to claim 110, wherein said one or more additional therapeutic agents are selected from the group consisting of: anti-viral agents, immunomodulators and immunostimulators.

112. A kit comprising a container comprising an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9, and a pharmaceutically acceptable carrier therefor.

113. (Canceled)

114. The kit according to claim 112, further comprising another anti-viral agent, an immunomodulator or an immunostimulator, or any combination thereof.

115. A method for treating a subject with an HIV-1 infection comprising the step of administering an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

116. A method for preventing or inhibiting HIV-1 infection in a subject comprising the step of administering an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

117. (Canceled)

118. A method for inhibiting HIV-1 virus binding to a T cell comprising the step of contacting said virus with an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

119. A method for inhibiting HIV-1 virus infection of a T cell comprising the step of contacting said virus with an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

120. A method of inhibiting HIV-1 gp120-mediated binding comprising the step of contacting a gp120-expressing HIV-1 virus with an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

121. The method according to any one of claims 115 or 116, further comprising the step of administering one or more additional therapeutic agents.

122. The method according to claim 121, wherein said one or more therapeutic agents are selected from the group consisting of: anti-viral agents, immunomodulators and immunostimulators.

123. The method according to any one of claims 115 or 116, wherein said administering step is performed via an intravenous, subcutaneous, intramuscular, oral, pulmonary inhalation, transdermal or parenteral route.

124-132. (Canceled)

133. A method for identifying a region of HIV-1 gp120 for use as an HIV-1 vaccine comprising the steps of:
a) producing in a non-human mammal a human monoclonal antibody and isolating said human monoclonal antibody that binds gp120 and that has neutralizing activity for HIV-1; and
b) identifying an epitope on said gp120 that is bound by said antibody.

134. (Canceled)

135. An isolated cell line that produces an isolated human antibody or antigen-binding portion thereof selected from the group consisting of
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said epitope is dependent on the presence of a sequence in the V1 loop;
an isolated human monoclonal antibody produced by the hybridoma cell line selected from the group consisting of: cell line 35D10/D2 (ATCC Accession Number PTA-3001), cell line 40H2/C7 (ATCC Accession Number PTA-3006), cell line 43A3/E4 (ATCC Accession Number PTA-3005), cell line 43C7/B9 (ATCC Accession Number PTA-3007), cell line 45D1/B7 (ATCC Accession Number PTA-3002), cell line 46E3/E6 (ATCC Accession Number PTA-3008), cell line 58E1/B3 (ATCC Accession Number PTA-3003), cell line 64B9/A6 (ATCC Accession Number PTA-3004), cell line 8.22.2 (ATCC Accession Number PTA-4007), cell line 8E11/A8 (ATCC Applicaton No. 10/628,004 Second Preliminary amendment dated Feb. 11, 2004 Accession Number PTA-4012), and cell line 8.27.3 (ATCC Accession Number PTA-3009), or an antigen-binding portion thereof;
an isolated human antibody or antigen-binding portion thereof that specifically binds to HIV-1 gp120 protein and that has HIV-1 neutralizing activity, wherein said antibody or antigen-binding portion thereof recognizes a epitope on a V1/V2 domain of HIV-1 gp120, wherein said antibody or antigen binding portion thereof recognizes a linear epitope on a V2 domain of HIV-1 gp120; and
an isolated human monoclonal antibody or antigen-binding portion thereof that specifically binds to an epitope on a V3 region of HIV-1 gp120, wherein said antibody binds to an epitope on the V3 region of HIV-1, and wherein said antibody does not specifically bind to a peptide consisting of SEQ ID NO: 9.

136. The cell line according to claim 135 that is a hybridoma.

137. The hybridoma cell line according to claim 136 that produces an antibody selected from the group consisting of 35D10/D2, secreted by a hybridoma designated by ATCC Accession Number PTA-3001, 40H2/C7, secreted by a hybridoma designated by ATCC Accession Number PTA-3006, 43A3/E4, secreted by a hybridoma designated by ATCC Accession Number PTA-3005, 43C7/B9, secreted by a hybridoma designated by ATCC Accession Number PTA-3007, 45D1/B7, secreted by a hybridoma designated by ATCC Accession Number PTA-3002, 46E3/E6, secreted by a hybridoma designated by ATCC Accession Number PTA-3008, 58E1/B3 secreted by a hybridoma designated by ATCC Accession Number PTA-3003, 64B9/A6, secreted by a hybridoma designated by ATCC Accession Number PTA-3004, 8E11/A8 secreted by a hybridoma designated by ATCC Accession Number PTA-4012, 8.27.3, secreted by a hybridoma designated by ATCC Accession Number PTA-3009 and 8.22.2, secreted by a hybridoma designated by ATCC Accession Number PTA-4007.

138. A non-human mammal expressing a human antibody that specifically binds HIV-1 gp120.

139. The human antibody according to claim 1 that competes with an antibody according to claim 20 for binding to an antigen bound by an antibody according to claim 20.


SEQUENCE LISTING (28 sequences)

SEQ ID NO: 1 - protein, 93 aa, Human immunodeficiency virus type 1
LKPCVKLTPLCVTLHCTNLKNATNTKSSNWKEMDRGEIKNCSFKVTTSIRNKMQKEYALFYKLDVVPIDNDNTSYKLINCNTSVITQACPKVS

SEQ ID NO: 2 - protein, 15 aa, Human immunodeficiency virus type 1
STNLKNATNTKSSNW

SEQ ID NO: 3 - protein, 15 aa, Artificial Sequence
  Description of Artificial Sequence Synthetic peptide
NTKSSNWKEMDGEIK

SEQ ID NO: 4 - protein, 17 aa, Human immunodeficiency virus type 1
TTSIRDKVQKEYALFYK

SEQ ID NO: 5 - protein, 35 aa, Human immunodeficiency virus type 1
CTRPNNNTRKSITIGPGRAFYATGDIIGDIRQAHC

SEQ ID NO: 6 - protein, 35 aa, Human immunodeficiency virus type 1
CTRPSNNTRKSIHIGPGRAFYTTGEIIGDIRQAHC

SEQ ID NO: 7 - protein, 33 aa, Human immunodeficiency virus type 1
TRPNYNKRKRIHIGPGRAFYTTKNIIGTIRQAH

SEQ ID NO: 8 - protein, 35 aa, Human immunodeficiency virus type 1
CTRPNYNKRKRIHIGPGRAFYTTKNIIGTIRQAHC

SEQ ID NO: 9 - protein, 20 aa, Human immunodeficiency virus type 1
CTRPNYNKRKRIHIGPGRAF

SEQ ID NO: 10 - protein, 20 aa, Human immunodeficiency virus type 1
RIHIGPGRAFYTTKNIIGTI

SEQ ID NO: 11 - protein, 20 aa, Human immunodeficiency virus type 1
YTTKNIIGTIRQAHCNISRA

SEQ ID NO: 12 - protein, 24 aa, Artificial Sequence
  Description of Artificial Sequence Synthetic peptide
YNKRKRIHIQRGPGRAFYTTKNII

SEQ ID NO: 13 - protein, 34 aa, Human immunodeficiency virus type 1
TRPNNNTRKSIRIQRGPGRAFVTTGKIGNMRQAH

SEQ ID NO: 14 - DNA, 32 bp, Artificial Sequence
  Description of Artificial Sequence Primer
agacatctagaatgagagtgaaggggatcagg

SEQ ID NO: 15 - DNA, 32 bp, Artificial Sequence
  Description of Artificial Sequence Primer
gctccgaattcttattatcttttttctctctg

SEQ ID NO: 16 - protein, 8 aa, Human immunodeficiency virus type 1
KEMDGEIK

SEQ ID NO: 17 - protein, 4 aa, Human immunodeficiency virus type 1
GPGR

SEQ ID NO: 18 - protein, 66 aa, Human immunodeficiency virus type 1
HCTNLKNATNTKSSNWKEMDRGEIKNCSFKVTTSIRNKMQKEYALFYKLDVVPIDNDNTSYKLINC

SEQ ID NO: 19 - protein, 78 aa, Human immunodeficiency virus type 1
NCIDLRNATNATSNSNTTNTTSSSGGLMMEQGEIKNCSFNITTSIRDKVQKEYALFYKLDIVPIDNPKNSTNYRLISC

SEQ ID NO: 20 - protein, 66 aa, Human immunodeficiency virus type 1
NCVKDVNATNTTNDSEGTMERGEIKNCSFNITTSIRDEVQKEYALFYKLDVVPIDNNNTSYRLISC

SEQ ID NO: 21 - protein, 72 aa, Human immunodeficiency virus type 1
NCTDLRNATNGNDTNTTSSSRGMVGGGEMKNCSFNITTNIRGKVQKEYALFYKLDIAPIDNNSNNRYRLISC

SEQ ID NO: 22 - protein, 67 aa, Human immunodeficiency virus type 1
KCTDLKNDTNTNSSSGRMIMEKGEIKNCSFNISTSIRGKVQKEYAFFYKLDIIPIDNDTTSYKLTSC

SEQ ID NO: 23 - protein, 72 aa, Human immunodeficiency virus type 1
NCTDLRNTTNTNNSTANNNSNSEGTIKGGEMKNCSFNITTSIRDKMQKEYALLYKLDIVSINDSTSYRLISC

SEQ ID NO: 24 - protein, 71 aa, Human immunodeficiency virus type 1
NCTDLGKATNTNSSNWKEEIKGEIKNCSFNITTSIRDKIQKENALFRNLDVVPIDNASTTTNYTNYRLIHC

SEQ ID NO: 25 - protein, 10 aa, Human immunodeficiency virus type 1
YTTKNIIGTI

SEQ ID NO: 26 - protein, 9 aa, Human immunodeficiency virus type 1
QKEYALFYK

SEQ ID NO: 27 - protein, 35 aa, Artificial Sequence
  Description of Artificial Sequence  Synthetic peptide
STRPSNNTRKSIHIGPGRAFYTTGEIIGDIRQAHC

SEQ ID NO: 28 - protein, 6 aa, Artificial Sequence
  Description of Artificial Sequence 6 His tag
HHHHHH